(19) United States
(12) Patent Application Publication
(10) Pub. No.: US 2022/0341931 A1
(43) Pub. Date: Oct. 27, 2022

(54) METHODS FOR ANTIBODY IDENTIFICATION AND QUANTIFICATION

(71) Applicant: SeqTech Diagnostics LLC, Nashville, TN (US)

(72) Inventor: Adam Robert PLATT, Whites Creek, TN (US)

(21) Appl. No.: 17/722,187

(22) Filed: Apr. 15, 2022

Related U.S. Application Data
(60) Provisional application No. 63/175,483, filed on Apr. 15, 2021.

(51) Int. Cl. G01N 33/569; C12N 15/10; C12Q 1/6804

(52) U.S. Cl. CPC G01N 33/56983; C12N 15/1065; C12Q 1/6804; G01N 2333/165; G01N 2469/20

(57) ABSTRACT

The disclosure provides compositions and methods for identifying and/or quantifying a binding member in a sample.

### CROSS REFERENCE TO RELATED APPLICATIONS

This application claims priority to U.S. Provisional Application Ser. No. 63/175,483, filed on Apr. 15, 2021, which is incorporated by reference herein in its entirety for all purposes.

### FIELD OF THE INVENTION

Embodiments of the disclosure concern at least the fields of molecular biology, biochemistry, immunology, and medicine.

### SEQUENCE LISTING

The instant application contains a Sequence Listing which has been submitted electronically in ASCII format and is hereby incorporated by reference in its entirety. Said ASCII copy, created on Apr. 14, 2022, is named STDL_001_01US_SeqList_ST25 and is about 21 KB in size.

### BACKGROUND

Antibody (Ab) detection is a rapidly growing area of interest in infectious disease medicine, and particularly with respect to determining whether a subject has been infected, and whether the subject mounted a protective immune response to the infectious agent. Unfortunately, technical issues limit the reliability of current antibody testing procedures. First, current tests are not quantitative; they cannot differentiate between a patient with a robust immune response and a patient lacking sufficient antibodies to successfully fight off subsequent infection. Both patients would likely test positive, but only one may be immune. Secondly, current antibody tests suffer from accuracy issues. For example, different types of currently available SARS-CoV-2 tests may produce different results for the same patient sample, i.e., some tests will test positive while others will test negative. This inaccuracy and fluctuation between different tests contribute to difficulty in prescribing treatment. Third, current antibody tests are limited in the number of different antibodies that can be detected at once. Most current tests are specific for only one type of antibody; therefore, testing multiple antibodies would require multiple tests. Currently, a simple and reliable method of obtaining information on a patient's entire antibody population (immunome) does not exist.

Clearly, a need exists for a more accurate, quantitative, and comprehensive method for antibody testing. The methods and compositions of the disclosure meet this need by applying a more precise analytical technique to identify and quantify antibodies and other analytes.

### SUMMARY

In one aspect, the disclosure provides methods of identifying and/or quantifying, in a test sample, one or more binding members capable of binding to each of one or more targets, the methods comprising:

contacting a test sample comprising binding members with a composition comprising at least one barcode-linked antigen (BLA), wherein each BLA comprises a barcode moiety linked to an antigen of a target, wherein binding members present in the test sample bind to antigens of the BLAs, thereby forming a mixture comprising BLA/binding member complexes;

optionally, isolating the BLA/binding member complexes from the mixture; and

determining the identity and quantity of the barcode moiety of each isolated BLA/binding member complex,

wherein the identity of each barcode moiety indicates the identity of its linked antigen and/or target, and the quantity of each barcode moiety indicates the quantity of binding members present in the test sample that bind its linked antigen and/or target, thereby identifying and/or quantifying the binding members in the test sample capable of binding to each of one or more targets. In particular embodiments, the test sample is contacted with the composition comprising the BLA(s) under conditions and for a time sufficient for binding members present in the test sample to bind to antigens of the BLAs.

In some embodiments of methods of the disclosure, the test sample is a biological sample obtained from a subject. In some embodiments, the biological sample comprises serum, plasma, whole blood, saliva, mucus, or a combination thereof. In some embodiments, the subject is a human.

In some embodiments of methods of the disclosure, the binding members comprise antibodies.

In some embodiments of methods of the disclosure, at least one target is a pathogen. In some embodiments, the pathogen is a virus, a bacterium, a fungus, a protozoan, a worm, or a prion. In some embodiments, the virus is a SARS-CoV-2 virus.

In some embodiments of aspects of the disclosure, the composition comprises a pool of BLAs. In some embodiments, the pool of BLAs comprises BLAs comprising different antigens. In some embodiments, at least two BLAs comprise different antigens from the same target. In some embodiments, the BLAs comprising different antigens from the same target comprise identical or different barcode moieties. In some embodiments, at least two BLAs comprise different antigens from different targets. In some embodiments, BLAs comprising different antigens from different targets comprise different barcode moieties.

In some embodiments of aspects of the disclosure, at least one antigen comprises a polypeptide, or an immunogenic fragment, variant, or epitope thereof, and in some embodiments, at least one antigen comprises a polysaccharide.

In some embodiments of methods of the disclosure, the method further comprises separating the barcode moiety of each BLA/binding member complex from its linked antigen.

In some embodiments of methods of the disclosure, each antigen is attached to its linked barcode moiety by a bond or linker. In some embodiments, each antigen is attached to its linked barcode moiety by a reagent capable of conjugating an oligonucleotide to a polypeptide. In some embodiments, the bond or linker comprises a heterobifunctional linker. In some embodiments, the bond or linker comprises HyNic-4FB, Succinimidyl 6-hydrazinonicotinate acetone hydrazone (SANH), succinimidyl 4-(N-maleimidomethyl) cyclohexane-1-carboxylate (SMCC), or biotin-streptavidin. In some embodiments, the bond or linker comprises HyNic-4FB. In some embodiments, the bond or linker is cleavable or breakable.

In some embodiments of aspects of the disclosure, each barcode moiety comprises an oligonucleotide, a peptide, a nano-structure, a fluorescent bead, an isobaric mass tag, or a combination thereof.

In some embodiments of aspects of the disclosure, the BLA/binding member complexes are isolated using a labelled anti-human antibody. In some embodiments, the label is capable of attaching to a solid support. In some embodiments, the label is a biotin label and the solid support is magnetic.

In some embodiments of aspects of the disclosure, each barcode moiety comprises an oligonucleotide. In some embodiments, each oligonucleotide comprises a unique sequence, one or more primer binding sites, and, optionally, a cleavage site. In some embodiments, the antigen of each BLA is identifiable by the unique sequence. In some embodiments, the target from which the antigen of each BLA was derived is identifiable by the unique sequence. In some embodiments, each oligonucleotide is about the same length. In some embodiments, the base composition of each oligonucleotide is substantially the same as any other oligonucleotide. In some embodiments, the unique sequence of each oligonucleotide is generated by a computational method. In some embodiments, the unique sequence of each oligonucleotide is selected from a predetermined pool of unique sequences. In some embodiments, each unique sequence is discrete from any other unique sequence of the pool of unique sequences by Hamming distance of at least 2. In some embodiments, a unique sequence is assigned to each different antigen. In some embodiments, the cleavage site of each oligonucleotide comprises a restriction site for an endonuclease.

In some embodiments of methods of the disclosure, determining the identity and quantity of the oligonucleotide of each isolated BLA/binding member complex comprises:

amplifying the oligonucleotides with primers to generate one or more sequencing-ready libraries;

sequencing the one or more libraries to identify the sequence of each oligonucleotide; and

quantifying each oligonucleotide.

In some embodiments of methods of the disclosure, the method further comprises, prior to amplification, separating the oligonucleotide from the antigen of each isolated BLA/binding member complex. In some embodiments, the oligonucleotide is separated from the antigen by treatment with one or more restriction enzymes.

In some embodiments of methods of the disclosure, the method further comprises, prior to amplification, purifying the separated oligonucleotides to generate purified oligonucleotides.

In some embodiments of aspects of the disclosure, each primer used for amplification comprises a sequence complementary to the primer binding site of each oligonucleotide and an adaptor sequence compatible with the sequencing method.

In some embodiments of methods of the disclosure, the method further comprises contacting a negative sample control that does not comprise binding members with a BLA composition.

In some embodiments of methods of the disclosure, the method further comprises contacting a positive sample control comprising binding members with a BLA composition.

In another aspect, the disclosure provides a kit used to quantify binding members in a test sample, optionally according to any one of the methods of the disclosure, the kit comprising a first vessel comprising a composition comprising at least one barcode-linked antigen (BLA), wherein each BLA comprises a barcode moiety attached to an antigen of a target.

In some embodiments of kits of the disclosure, at least one target is a pathogen. In some embodiments, the pathogen is a virus, a bacterium, a fungus, a protozoan, a worm, or a prion. In some embodiments, the virus is a SARS-CoV-2 virus.

In some embodiments of the kits of the disclosure, the first vessel comprises a pool of BLAs. In some embodiments, the pool of BLAs comprises two or more BLAs comprising different antigens. In some embodiments, the different antigens are from the same target. In some embodiments, BLAs comprising different antigens from the same target comprise identical or different barcode moieties. In some embodiments, the different antigens are from different targets. In some embodiments, BLAs comprising different antigens from different targets comprise different barcode moieties.

In some embodiments of the kits of the disclosure, at least one antigen comprises a polypeptide, or an immunogenic fragment, variant, or epitope thereof, and in some embodiments, at least one antigen comprises a polysaccharide.

In some embodiments of the kits of the disclosure, the barcode moiety of each BLA/binding member complex can be separated from its linked antigen.

In some embodiments of the kits of the disclosure, the kit further comprises a second vessel comprising a reagent capable of separating the antigen from the attached barcode moiety of each BLA. In some embodiments, the reagent comprises a restriction enzyme.

In some embodiments of the kits of the disclosure, each antigen is attached to its linked barcode moiety by a bond or linker. In some embodiments, the bond or linker comprises a heterobifunctional linker. In some embodiments, the bond or linker comprises HyNic-4FB, Succinimidyl 6-hydrazinonicotinate acetone hydrazone (SANH), succinimidyl 4-(N-maleimidomethyl) cyclohexane-1-carboxylate (SMCC), or biotin-streptavidin. In some embodiments, the bond or linker comprises HyNic-4FB. In some embodiments, the bond or linker is cleavable or breakable.

In some embodiments of the kits of the disclosure, each barcode moiety comprises an oligonucleotide, a peptide, a nano-structure, a fluorescent bead, an isobaric mass tag, or a combination thereof.

In some embodiments of the kits of the disclosure, the kit further comprises a third vessel comprising a labelled agent capable of binding to the binding members. In some embodiments, the third vessel comprises labelled anti-human antibodies. In some embodiments, the label is capable of attaching to a solid support. In some embodiments, the label is a biotin label and the solid support is magnetic.

In some embodiments of the kits of the disclosure, the kit further comprises a fourth vessel comprising the solid support.

In some embodiments of the kits of the disclosure, each barcode moiety comprises an oligonucleotide. In some embodiments, each oligonucleotide comprises a unique sequence; one or more primer binding sites; and, optionally, a cleavage site. In some embodiments, the antigen of each BLA is identifiable by the unique sequence. In some embodiments, the target from which the antigen of each BLA was derived is identifiable by the unique sequence. In some embodiments, each oligonucleotide is about the same length. In some embodiments, the base composition of each oligonucleotide is substantially the same as any other oligonucleotide. In some embodiments, the unique sequence of each oligonucleotide is generated by a computational method. In some embodiments, the unique sequence of each oligonucleotide is selected from a predetermined pool of unique sequences. In some embodiments, each unique sequence is discrete from any other unique sequence of the pool of unique sequences by Hamming distance of at least 2. In some embodiments, a unique sequence is assigned to each different antigen. In some embodiments, the cleavage site of each oligonucleotide comprises a restriction site for an endonuclease. In some embodiments, each primer binding site comprises a sequence complementary to an amplification primer comprising an adaptor sequence compatible with a sequencing method.

In some embodiments of the kits of the disclosure, the kit further comprises a fifth vessel comprising said primer.

In some embodiments of the kits of the disclosure, the kit further comprises a sixth vessel comprising a negative sample control that does not comprise binding members.

In some embodiments of the kits of the disclosure, the kit further comprises a seventh vessel comprising a positive sample control comprising binding members.

In another aspect, the disclosure provides a composition comprising at least one barcode-linked antigen (BLA), wherein each BLA comprises a barcode moiety linked to an antigen of a target; wherein the barcode moiety can be quantified; and wherein the barcode moiety can identify its linked antigen. In some embodiments, at least one antigen is capable of binding to a binding member in a test sample. In some embodiments, the binding member comprises an antibody.

In some embodiments of the compositions of the disclosure, the test sample is a biological sample obtained from a subject. In some embodiments, the subject is a human. In some embodiments, the biological sample comprises serum, plasma, whole blood, saliva, mucus, or a combination thereof.

In some embodiments of the compositions of the disclosure, at least one antigen comprises a polypeptide, or an immunogenic fragment, variant, or epitope thereof. In some embodiments, at least one antigen comprises a polysaccharide.

In some embodiments of the compositions of the disclosure, the target is a pathogen. In some embodiments, the pathogen is a virus, a bacterium, a fungi, a protozoa, a worm, or a prion. In some embodiments, the virus is a SARS-CoV-2 virus.

In some embodiments of the compositions of the disclosure, the composition comprises a pool of BLAs. In some embodiments, the pool of BLAs comprises BLAs comprising different antigens. In some embodiments, at least two different antigens are from the same target. In some embodiments, BLAs comprising different antigens from the same target comprise identical barcode moieties. In some embodiments, the different antigens are from at least two different targets. In some embodiments of the compositions of the disclosure, BLAs comprising different antigens comprise different barcode moieties. In some embodiments, the barcode moiety can be separated from its linked antigen. In some embodiments of the compositions of the disclosure, each antigen is attached to its linked barcode moiety by a bond or linker. In some embodiments, each antigen is attached to its linked barcode moiety by a reagent capable of conjugating an oligonucleotide to a polypeptide. In some embodiments of the compositions of the disclosure, the bond or linker comprises a heterobifunctional linker. In some embodiments, the bond or linker comprises HyNic-4FB, Succinimidyl 6-hydrazinonicotinate acetone hydrazone (SANH), succinimidyl 4-(N-maleimidomethyl) cyclohexane-1-carboxylate (SMCC), or biotin-streptavidin. In some embodiments, the bond or linker comprises HyNic-4FB. In some embodiments, the bond or linker is cleavable or breakable. In some embodiments of the compositions of the disclosure, each barcode moiety comprises an oligonucleotide, a peptide, a nano-structure, a fluorescent bead, an isobaric mass tag, or a combination thereof.

In some embodiments of the compositions of the disclosure, each barcode moiety comprises an oligonucleotide. In some embodiments, each oligonucleotide comprises a unique sequence; one or more primer binding sites; and, optionally, a cleavage site. In some embodiments, the antigen of each BLA is identifiable by the unique sequence. In some embodiments, the target from which the antigen of each BLA was derived is identifiable by the unique sequence. In some embodiments, each oligonucleotide is about the same length. In some embodiments, the base composition of each oligonucleotide is substantially the same as any other oligonucleotide. In some embodiments, the unique sequence of each oligonucleotide is generated by a computational method. In some embodiments, the unique sequence of each oligonucleotide is selected from a predetermined pool of unique sequences. In some embodiments, each unique sequence is discrete from any other unique sequence of the pool of unique sequences by Hamming distance of at least 2. In some embodiments, a unique sequence is assigned to each different antigen.

In some embodiments of the compositions of the disclosure, the oligonucleotide can be separated from the antigen. In some embodiments, the oligonucleotide can be separated from the antigen by treatment with one or more restriction enzymes. In some embodiments, the cleavage site of each oligonucleotide comprises a restriction site for an endonuclease. In some embodiments, each primer binding site comprises a sequence complementary to an amplification primer comprising an adaptor sequence compatible with a sequencing method.

In a further aspect, the disclosure further provides a method of producing any one of the compositions of the disclosure, the method comprising attaching the barcode moiety to the antigen.

### BRIEF DESCRIPTION OF THE DRAWINGS

Various objects and advantages and a more complete understanding of the disclosure are apparent and more readily appreciated by reference to the following Detailed Description and to the appended claims when taken in conjunction with the accompanying Drawings wherein:

FIG. 1 is a schematic diagram depicting illustrative antigens of a SARS-CoV-2 virus and an illustrative structure of a barcode-linked antigen (BLA).

FIG. 2 is a schematic diagram depicting an illustrative process of antibodies (Abs) binding to the BLA structures. In this example, Antibody “X” (white) binds to “X” BLAs, Antibody “Y” (checkered) binds to “Y” BLAs, and Antibody “Z” (black) binds to “Z” BLAs.

FIG. 3 is a schematic diagram depicting step 1 of an illustrative workflow comprising: mixing a patient sample comprising patient antibodies with a pool of BLAs to generate a first mixture comprising “X” and “Z” BLA/patient antibody complexes, “Y” unbound BLAs, and excess “X” and “Z” unbound BLAs.

FIG. 4 is a schematic diagram depicting step 2 of an illustrative workflow comprising: adding anti-human antibodies with a biotin linker (anti-human antibodies depicted in grey) to the mixture from step 1 to generate a second mixture comprising “X” and “Z” BLA/patient antibody/anti-human antibody complexes, unbound anti-human antibodies, “Y” unbound BLAs, and excess “X” and “Z” unbound BLAs.

FIG. 5 is a schematic diagram depicting step 3 of an illustrative workflow comprising: adding streptavidin-coated magnetic beads to the second mixture from step 2 to generate a third mixture comprising “X” and “Z” BLA/patient antibody/anti-human antibody/beads complexes, anti-human antibody/beads complexes, excess unbound beads, “Y” unbound BLAs, and excess “X” and “Z” unbound BLAs.

FIG. 6 is a schematic diagram depicting step 4 of an illustrative workflow comprising: applying a magnetic force to the third mixture from step 3 to separate the BLAs that were not bound to antibodies from the BLAs that were bound to antibodies.

FIG. 7 is a schematic diagram depicting step 5 of an illustrative workflow comprising: removing the BLAs that were not bound to antibodies to generate a “negative pool” comprising the BLAs not bound to antibodies, including the “Y” unbound BLAs and the excess “X” and “Z” unbound BLAs. Simultaneously, a “positive pool” comprising “X” and “Z” BLA/patient antibody/anti-human antibody/beads complexes, anti-human antibody/beads complexes, and excess unbound beads is generated.

FIG. 8 is a schematic diagram depicting step 6 of an illustrative workflow comprising: treating both pools from step 5 with one or more restriction enzymes to separate the barcode from the antigen of each BLA.

FIG. 9 is a schematic diagram depicting step 7 of an illustrative workflow comprising: filtering the positive and negative pools to purify the separated barcodes.

FIG. 10 is a graph showing the high level of linearity achieved for anti-nucleocapsid antibody quantification using methods and compositions of the disclosure.

FIG. 11 is a graph showing the high level of linearity achieved for anti-spike antibody quantification using methods and compositions of the disclosure.

FIG. 12 is a graph showing an example using methods and compositions of the disclosure for diagnostic purposes.

FIG. 13 provides a schematic illustration of an Index Plate used for sequencing captured barcodes and the reagents in each well.

FIG. 14 provides a bar graph showing barcode sequence counts for each BLA in an 8-BLA system. For each of the four sets of barcode counts, the BLA from left to right corresponds to the order shown from top to bottom in the figure legend on the right of the graph.

FIG. 15 is a graph showing the high level of linearity achieved for anti-spike antibody quantification using normalized Spike/Nuc ratios.

### DETAILED DESCRIPTION

As described in the Background section, numerous problems exist with current qualitative antibody tests. Many of these problems stem from the inherent limitations of the enzyme-linked immunosorbent assay (ELISA) assay, which uses an enzyme-mediated photochemical reaction (e.g., a color change) to signal a positive result. In ELISA assays, the presence of the tested antibody triggers a chain reaction that is designed to run to completion regardless of the number of antibodies. Thus, a small number of antibodies could produce the same color change (test result) on an ELISA test as a large number of antibodies. This chain reaction can also be inhibited or falsely initiated, leading to issues with the test's accuracy and reliability.

Instead of using antigens linked with enzymes as with current ELISA-based methods, the disclosure provides compositions and methods using antigens linked with countable “barcodes,” i.e., barcode-linked antigens (BLAs). The compositions and methods of the disclosure are associated with numerous benefits, including but not limited to precise quantitation, high-throughput capability, and simple bioinformatics requirements.

In some embodiments, a linear relationship exists between the number of binding members (e.g., antibodies) present in a test sample and the amount of positive signal generated from the barcodes of BLAs that bind to said binding members. In some embodiments, each type of antigen is labelled with its own unique barcode. In some embodiments, BLAs that bind to antibodies are isolated and the barcodes from the isolated BLAs are analyzed to identify and quantify the binding members present in the sample. In some embodiments, identification and quantification of binding members is achieved via a sequencing-by-synthesis (SBS) method. For example, in some embodiments, within each individual SBS reaction a single DNA oligo is analyzed and each of these reactions are separately recorded, allowing for precise detection of each oligo. The ability to process millions of oligos in parallel allows the sequencer to sample the oligo population at great depth with wide dynamic range. This ability enables the instrument to simultaneously measure both rare variants within the population and its most common species without distorting the quantitative relationship between the two. This approach provides a direct, linear relationship between the number of binding members and the amount of positive signal generated by the assay.

Some embodiments of the methods and compositions of the disclosure enable precise quantification of hundreds of different antibodies simultaneously within a single test.

The current generation of DNA sequencers use the Sequencing-by-Synthesis (SBS) process to analyze millions of short pieces of DNA at once. This is due to a limitation in the technique: it is only able to analyze short segments of DNA, ˜100s of bases at a time. Therefore, methods utilizing these SBS DNA sequencers need to first fragment the DNA sample into thousands of smaller fragments and analyze them in thousands of parallel reactions before using computational methods to reconstruct the smaller fragments into longer DNA molecules. The short read length is viewed by most as the main limitation of SBS. Current sequencing methods compensate for short read length by increasing throughput, i.e. the number of SBS reactions per sequencing run. This increase in reactions per run allowed for a vast expansion in sequencing throughput capacity despite the short-read limitation.

In some embodiments, the methods and compositions of the disclosure utilize this “limitation” of SBS methods to provide precise and high-throughput identification and quantification of binding members. Because the goal of the methods of the disclosure is not to sequence long chains of DNA as in most next-generation sequencing applications, but rather to detect a large population of short DNA oligos, current sequencers can provide robust analytical power. For example, a MiSeq DNA sequencer (Illumina) with a relatively low throughput capacity can perform 50 million SBS reactions at once, while top-of-the-line sequencers can reach a throughput at 100 times that of MiSeq, allowing billions of separate reactions to occur simultaneously. This ultra-high-throughput capability in turn allows a nearly unlimited number of different binding members (such as antibodies) to be tested in a single assay. For example, in some embodiments, the compositions and methods of the disclosure may be used to test thousands of antigens and/or viruses simultaneously, allowing physicians to screen patient samples for every antibody species extant.

For a myriad of next-generation sequencing applications that use SBS, complicated algorithms are required to analyze sequencing results. As described above, because a goal of most sequencing applications is to recreate the sequence of longer molecules from the short reads, such algorithms need to address complex issues in sequence alignment, bias correction, error calling, etc. Developing advanced algorithms that provide more accurate sequencing results remains a difficult task in the bioinformatics field. In contrast, the methods of the disclosure require much simpler bioinformatics analysis that can be completed by a simple algorithm. In some embodiments, oligos are sorted into groups with a group for each oligo sequence found in the population. Each different oligo sequence indicates the identity of its originally linked antigen. The number of oligos in each group can then be counted to indicate the number of their originally linked antigen, which directly reflects the number of binding members in the test sample without the need for complicated bioinformatics analysis.

The methods and compositions of the disclosure provide a new arena of diagnostic testing in which a patient's entire immune system (e.g., every antibody or known type of antibody) can be determined. In certain embodiments, the patient's entire immune system, e.g., all antibodies or known types of antibodies, can be monitored over time allowing for strategic treatment options. Quantitative and regular testing using methods of the disclosure allow antibody levels to be tracked throughout a patient's lifetime, providing an “antibody fingerprint” for each individual patient.

The accurate quantification methods of the disclosure also allow reliable detection of antibody levels against one or more pathogens in disease prevention, thus providing more precise vaccination regimens to maintain immunity.

In some embodiments, the disclosure provides one or more internal controls used in antibody testing, which may offer additional advantages to the methods and compositions of the disclosure. Because each individual may possess different levels of a tested antibody, internal controls may provide “immune benchmarks” that allow normalization of the tested antibody levels within the context of an individual's immune system.

In some embodiments, the one or more internal controls is used to quantify a tested antibody in an individual at a given time. In some embodiments, the ratio [level of the tested antibody]:[level of the one or more internal controls] indicates a diagnostic result for the individual's immunity against the pathogen corresponding to the tested antibody.

In some embodiments, the one or more internal controls is used to quantify a tested antibody in an individual over a period of time. In some embodiments, the change in the ratio [level of the tested antibody]:[level of the one or more internal controls] over a period of time indicates a diagnostic result for the individual's immunity against the pathogen corresponding to the tested antibody. For example, a [level of the tested antibody]:[level of the one or more internal controls] ratio of 0.2 measured on Day 1 and a ratio of 0.15 measured on Day 2 would indicate a drop in the level of the tested antibody in an individual over a period of two days.

In some embodiments, the tested antibody is an antibody against SARS-COV-2 and the one or more internal controls comprise an antigen derived from a non-SARS-COV-2 pathogen.

In some embodiments, the one or more internal controls comprise an antigen derived from a pathogen selected from Influenza A, Influenza B, Varicella-Zoster Virus, Human Metapneumovirus (hMPV), Human cytomegalovirus (HCMV), and Epstein-Barr virus (EBV).

In some embodiments, the one or more internal controls comprise an antigen, or antigenic fragment thereof, selected from Influenza A Hemagglutinin (HA) and Influenza B HA. In some embodiments, the one or more internal controls comprise an antigen, or antigenic fragment thereof, that binds to antibodies generated by a flu vaccine.

In some embodiments, the one or more internal controls comprise an antigen, or antigenic fragment thereof, selected from Varicella-Zoster Virus gE/gI, hMPV B Glycoprotein G, HCMV Glycoprotein B/gB Protein, and EBV Glycoprotein gp350.

In some embodiments, the one or more internal controls comprise Influenza A HA, Influenza B HA, Varicella-Zoster Virus gE/gI, hMPV B Glycoprotein G, HCMV Glycoprotein B/gB Protein, and EBV Glycoprotein gp350, or antigenic fragments thereof. Methods, Compositions, and Kits

The present invention is described more fully hereinafter using illustrative, non-limiting embodiments, and references to the accompanying figures. This invention may, however, be embodied in many different forms and should not be construed as to be limited to the embodiments set forth below.

In certain embodiments, the disclosure provides methods for identifying and/or quantifying one or more binding members in a test sample, based on the ability of the binding member to specifically bind a target, e.g., an antigen. The methods may be practiced to determine the presence or absence of a binding member in a test sample, or to quantitate the amount of a binding member in a sample. The number of different targets and their identities may be varied, so the assay may be used to identify and/or quantitate a wide variety of different binding members, and may be used to identify and/or quantitate a single binding member or multiple binding members in a test sample.

To assist in the identification and/or quantification of the binding members, the targets to which they bind are coupled to a unique identifier (barcode), and the unique identifiers associated with the targets bound by the binding members determined and used to characterize the binding members.

In particular embodiments, the methods comprise:

contacting a test sample with at least one barcode-linked antigen (BLA), wherein each BLA comprises a barcode moiety linked to a target, or fragment thereof, and

determining the identity and/or quantity of the barcode moieties present within any resulting complexes of binding members and targets;

wherein the identity of each barcode moiety indicates the identity of its linked target, and wherein the quantity of each barcode moiety indicates the quantity of binding members present in the test sample that bind to the linked target,

thereby identifying and/or quantifying the binding members in the test sample capable of binding to each of one or more targets.

In particular embodiments, test samples are contacted with BLAs, wherein binding members present in the test sample bind to the target regions of the BLAs, thereby forming a mixture comprising BLA/binding member complexes. In particular embodiments, the test samples are contacted with the BLAs under conditions and for a time sufficient for binding members present in the test sample to bind to the target regions of the BLAs. Such conditions and time are known and may readily be determined, e.g., based on the test sample and BLA. In certain embodiments, the conditions may be those described in the accompanying examples or physiological conditions. In certain embodiments, the time may be one minute or less, 5 minutes or less, 30 minutes or less, or one hour or less. In certain embodiments, the BLA/binding member complexes are isolated from or separated from unbound binding members and BLAs before determining the identity and/or quantity of the barcode moieties present within the complexes.

The compositions and methods disclosed herein are advantageous in both identifying and/or quantitating a binding member in a sample and may be used on a variety of different types of samples that contain binding members. For example, test samples may include biological samples, environmental samples, or other types of samples.

In some embodiments, the test sample is a biological sample, such as a biological sample obtained from a subject, such as, e.g., serum, plasma, whole blood, saliva, mucus, or a combination thereof. In some embodiments, the subject is a human. In some embodiments, the biological sample comprises antibodies, cells, and/or other biological molecules, including certain binding members that are biological molecules capable of binding to a target or antigenic fragment thereof. In some embodiments, the biological sample comprises at least one immunoglobulin that can bind to at least one antigen of a target, e.g., an infectious agent. In some embodiments, the immunoglobulin may be IgG.

In particular embodiments, binding members are antibodies present within a biological sample obtained from a subject, such as a subject previously or currently infected by an infectious agent or suspected of being previously or currently infected by an infectious agent. Accordingly, in certain embodiments, the methods are practiced to identify and/or quantitate one or more binding member in a biological sample comprising antibodies, such as blood or serum.

The compositions and methods disclosed may be used to quantitate and/or identify binding members within a sample. As such, binding members may be considered a type of analyte. As used in the disclosure, “binding members” may refer to an agent that is capable of binding to a target, e.g., to an antigen or antigenic fragment of a target. Illustrative examples of binding members include, but are not limited to: proteins, lipids, polysaccharides, nucleic acids, and cells. In some embodiments, the binding member is an antibody. In some embodiments, the binding member is a T cell or B cell. In some embodiments, the binding member is a pathogen. In some embodiments, the binding member is a virus. In some embodiments, the binding member is a bacterial cell.

In some embodiments, the binding member is an antibody. Each type of antibody may have a different molecular arrangement in the Hypervariable Regions of its structure. Each Hypervariable Region may a high affinity for only a certain molecular structure or antigen. Antibodies with the same molecular structure in their hypervariable region and that bind the same antigen structure may be referred to in the disclosure as being of the same “species” of antibodies. Antibodies of a different species may bind different antigens. Similarly, binding members that bind the same antigen structure may be referred to in the disclosure as being of the same species of binding members. As used in the disclosure, a different antibody may refer to an antibody of a different species, or a different antibody molecule of the same species; a different binding member may refer to a binding member of a different species, or a different binding member molecule/cell/particle of the same species.

The compositions and methods disclosed herein may be used to identify and/or quantitate binding members that bind to a wide variety of different targets. The methods may be practiced to identify and/or quantitate binding members that bind to one target or to multiple targets. In some embodiments, the target is a pathogen or an infectious agent. In some embodiments, the target is a virus, a bacterium, a fungi, a protozoa, a worm, or a prion. In certain embodiments, the methods may be practiced to determine whether a subject has been infected by one or more pathogen or infectious agent, either previously infected or currently infected, and to identify the one or more pathogen and/or infectious agent with which the subject has been infected. In particular embodiments, the binding members being assayed are antibodies produced by the subject that bind to the one or more pathogens and/or infectious agents, and the robustness of the subject's immune response to each of the one or more pathogens and/or infectious agents may be determined by quantifying the binding members that bind to each of the pathogens and/or infectious agents.

Illustrative examples of bacterial species that can be the target of the compositions and methods of the disclosure include, but are not limited to: a Mycobacterium spp., a Pneumococcus spp., an Escherichia spp., a Campylobacter spp., a Corynebacterium spp., a Clostridium spp., a Streptococcus spp., a Staphylococcus spp., a Pseudomonas spp., a Shigella spp., a Treponema spp., a Vibrio spp., a Neisseria spp., a Klebsiella spp., a Citrobacter spp., a Hafnia spp., a Proteus spp., a Haemophilus spp., or a Salmonella spp.

Illustrative examples of fungal species that can be the target of the compositions and methods of the disclosure include, but are not limited to: an Aspergillus spp., a Blastomyces spp., a Candida spp., a Coccidioides spp., a Cryptococcus spp., dermatophytes, a Tinea spp., a Trichophyton spp., a Microsporum spp., a Fusarium spp., a Histoplasma spp., a Mucoromycotina spp., a Pneumocystis spp., a Sporothrix spp., an Exserohilum spp., or a Cladosporium spp.

Illustrative examples of viruses that can be the target of the compositions and methods of the disclosure include, but are not limited to: Influenza A such as H1N1, H1N2, H3N2 and H5N1 (bird flu), Influenza B, Influenza C virus, Hepatitis A virus, Hepatitis B virus, Hepatitis C virus, Hepatitis D virus, Hepatitis E virus, Rotavirus, any virus of the Norwalk virus group, enteric adenoviruses, parvovirus, Dengue fever virus, Monkey pox, Mononegavirales, Lyssavirus such as rabies virus, Lagos bat virus, Mokola virus, Duvenhage virus, European bat virus 1 & 2 and Australian bat virus, Ephemerovirus, Vesiculovirus, Vesicular Stomatitis Virus (VSV), Herpesviruses such as Herpes simplex virus types 1 and 2, varicella zoster, cytomegalovirus, Epstein-Bar virus (EBV), human herpesviruses (HHV), human herpesvirus type 6 and 8, Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV), HIV (human immunodeficiency virus; including HIV type 1, and HIV type 2), visna-maedi virus (VMV) virus, the caprine arthritis-encephalitis virus (CAEV), equine infectious anemia virus (EIAV), feline immunodeficiency virus (FIV), bovine immune deficiency virus (BIV), and simian immunodeficiency virus (SIV), papilloma virus, murine gammaherpesvirus, Arenaviruses such as Argentine hemorrhagic fever virus, Bolivian hemorrhagic fever virus, Sabia-associated hemorrhagic fever virus, Venezuelan hemorrhagic fever virus, Lassa fever virus, Machupo virus, Lymphocytic choriomeningitis virus (LCMV), Bunyaviridiae such as Crimean-Congo hemorrhagic fever virus, Hantavirus, hemorrhagic fever with renal syndrome causing virus, Rift Valley fever virus, Filoviridae (filovirus) including Ebola hemorrhagic fever and Marburg hemorrhagic fever, Flaviviridae including Kaysanur Forest disease virus, Omsk hemorrhagic fever virus, Tick-borne encephalitis causing virus and Paramyxoviridae such as Hendra virus and Nipah virus, variola major and variola minor (smallpox), alphaviruses such as Venezuelan equine encephalitis virus, eastern equine encephalitis virus, western equine encephalitis virus, SARS-associated coronavirus (SARS-CoV), severe acute respiratory syndrome coronavirus 2 (SARS-CoV-2), West Nile virus, and any encephalitis causing virus.

Illustrative examples of protozoa species that can be the target of the compositions and methods of the disclosure include, but are not limited to: Cryptosporidium spp., Cyclospora spp., Cystoisospora spp., Entamoeba spp., Giardia spp., Trypanosoma spp., Plasmodium spp., and Toxoplasma spp.

In certain embodiments, the compositions and methods of the disclosure may be used to test for binding members for one, more than 1, more than 2, more than 5, more than 10, more than 50, more than 100, more than 200, more than 500, more than 1000, more than 2000, more than 3000, more than 4000, more than 5000, more than 6000, more than 7000, more than 8000, more than 9000, more than 10,000, more than 20,000, more than more than 30,000, or more than 40,000, antigens and/or targets simultaneously using the same pool of BLAs, in one or more assays disclosed herein. In particular embodiments, the compositions and methods of the disclosure may be used to test for binding members for one to 100,000, one to 50,000, one to 40,000, one to 20,000, one to 10,000, one to 1,000, one to 100, or one to 10 antigens and/or targets simultaneously.

### Barcode-Linked Antigens (BLAs)

Methods disclosed herein are practiced using barcode-linked antigens (BLAs), wherein each BLA comprises a barcode moiety linked to a target or fragment thereof; wherein the barcode moiety can be quantified; and wherein the barcode moiety can identify its linked target. In particular embodiments, the target is a fragment of a complete target, wherein the fragment is bound by the binding member that binds the complete target. For example, the target moiety present in the BLA may be an antigenic region or epitope of a target, such as a polypeptide comprising an epitope of a target pathogen or infectious agent that is bound by antibodies (binding members) produced by the subject in response to infection by the target pathogen or infectious agent. One such example is a polypeptide comprising an antigenic epitope of a SARS-CoV-2 spike protein.

### Antigens and Targets

As used herein in the context of the BLA, the term “antigen” can include a variety of different molecules (targets or fragments thereof) that may be bound by a binding member. In some embodiments, at least one antigen of the BLA is capable of binding to a binding member in a test sample. In certain embodiments, an antigen may refer to a pathogen, infectious agent, or toxin or other foreign substance (or an antigenic region of any of these) that induces or is capable of inducing an immune response in a subject contacted with the antigen. In some embodiments, the antigen induces production of antibodies specific for the target. Antigens that bind the same antibody species may be referred to in the disclosure as being of the same species of antigens. BLAs having antigens that bind the same antibody species may be referred to in the disclosure as being of the same species of BLAs. A different antigen may refer to an antigen of a different species, or a different antigen molecule/particle of the same species. A different BLA may refer to a BLA of a different species, or a different BLA molecule/partible of the same species.

In some embodiments, the antigens of the BLAs are capable of differentiating patients who generated antibodies from a prior infection from patients who generated antibodies from a prior vaccination. For example, using both the spike protein antigen and nucleocapsid antigen from the SARS-CoV-2 virus in BLAs would identify patients who generated antibodies from a SARS-CoV-2 infection, because current SARS-CoV-2 vaccines only stimulate production of antibodies against the spike protein. Thus, a biological sample from a patient who had generated antibodies from a previous SARS-CoV-2 infection would contain antibodies that bound both spike protein and nucleocapsid antigens, whereas a biological sample from a patient who had only generated antibodies from a previous vaccination would contain antibodies that bound the spike protein antigen but not the nucleocapsid antigen.

In some embodiments, the antigens of the BLAs are capable of differentiating between various strains of an infectious agent, such as between different strains of SARS-CoV-2, by using antigens specific for the different strains, such as spike protein antigens specific to the original, UK, Brazil and South Africa variants of SARS-CoV-2.

In some embodiments, the antigen of the BLA comprises a polypeptide, or an immunogenic fragment, variant, or epitope thereof. In some embodiments, the antigen of the BLA comprises a surface protein of a virus, or an immunogenic fragment, variant, or epitope thereof. In some embodiments, the antigen of the BLA comprises a spike protein, a membrane glycoprotein, or an envelope protein of a virus, or an immunogenic fragment, variant, or epitope thereof. In some embodiments, the antigen of the BLA comprises a capsid protein of a virus. In some embodiments, the antigen of the BLA comprises a nucleocapsid protein of a virus.

Antigens (and immunogenic fragments, variants, and epitopes thereof) are immunogenic when they are capable of being bound by an antibody, e.g., an antibody specific for the antigen, and/or inducing an immune response, e.g., in vivo. Antigens may comprise epitopes, e.g., smaller regions of the antigen that are recognized or bound by an antibody that binds the antigen. In certain embodiments, an immunogenic fragment consists of less than 90%, less than 80%, less than 70%, less than 60%, less than 50%, less than 40%, less than 30%, less than 20%, or even less than 10% of the total, e.g., the full-length or native polypeptide. In certain embodiments, a variant of a polypeptide or nucleic acid antigen or fragment thereof comprises one or more amino acid or nucleotide modification as compared to the native or wild-type polypeptide or nucleic acid reference sequence, e.g., one or more amino acid or nucleotide substitutions, deletions, and/or insertions. In particular embodiments, the variant has at least 80%, at least 85%, at least 90%, at least 95%, at least 98%, or at least 99% sequence identity to the native or wild-type polypeptide or nucleic acid reference sequence. As used herein, the terms “identity” and “identical” refer, with respect to a polypeptide or polynucleotide sequence, to the percentage of exact matching residues in an alignment of that “query” sequence to a “subject” sequence, such as an alignment generated by the BLAST algorithm. Identity is calculated, unless specified otherwise, across the full length of the subject sequence. Thus, a query sequence “shares at least x % identity to” a subject sequence if, when the query sequence is aligned to the subject sequence, at least x % (rounded down) of the residues in the subject sequence are aligned as an exact match to a corresponding residue in the query sequence. Where the subject sequence has variable positions (e.g., residues denoted X), an alignment to any residue in the query sequence is counted as a match. Sequence alignments may be performed using the NCBI Blast service (BLAST+version 2.12.0).

Protein antigens used in the disclosure may be recombinantly produced and purified according to any means or method known in the art. In certain embodiments, the protein antigens are recombinantly produced in cells, optionally Escherichia coli, yeast, insect, plant, or mammalian cells.

In some embodiments, the protein antigens may be recombinantly produced in bacterial host cells. The bacterial cells may allow genetic manipulation for insertion of a gene of interest, allow cultivation to high cell densities, and may be cultivated on a manufacturing scale. In some embodiments, the protein antigens may be recombinantly produced using E. coli (Lee, S. Y. High cell-density culture of Escherichia coli. Trends in Biotechnology, 1996, 14, 98-105; Hannig, G., and Makrides, S. O. Strategies for optimizing heterologous protein expression in Escherichia coli. Trends in Biotechnology, 1998, 16, 54-60), Bacillus subtilis, Pseudomonas fluorescens (Squires et al., Heterologous Protein Production in P. fluorescens. BioProcesss International, December 2004, 54-58), Corynebacterium (US20060003404A1) and Lactococcus lactis (Mierau et al., Industrial-scale production and purification of a heterologous protein in Lactococcus lactis using the nisin-controlled gene expression system NICE: The case of lysostaphin. Microbial Cell Factories, 2005, 4:15 doi:10.1 186/1475-2859-4-15). In some embodiments, the host cells used for antigen production are E. coli cells.

In some embodiments, the antigen of the BLA comprises a T lymphocyte dependent (TD) antigen. In some embodiments, the antigen of the BLA comprises a T lymphocyte independent (TI) antigen. In some embodiments, the antigen of the BLA comprises a polysaccharide. In some embodiments, said polysaccharide originates from a gram-positive or gram-negative bacterium. In some embodiments, said polysaccharide is a capsular polysaccharide.

Polysaccharide antigens used in the disclosure may be produced and purified according to any means or method known in the art. In certain embodiments, the polysaccharide antigens are produced by fermentation of a bacterial target comprising the polysaccharide antigens. For example, high yield production of bacterial polysaccharides may be achieved using the methods disclosed in PCT publication WO/2014/080423.

In some embodiments, the antigen of the BLA comprises a portion of the target that is predetermined, such as a known antigenic epitope within the target. In some embodiments, the antigen of the BLA comprises a portion of the target that is not predetermined.

In some embodiments, the antigen of the BLA comprises a molecule or molecular fragment from a lysed or fragmented target. In some embodiments, the antigen of the BLA comprises a molecule or molecular fragment from a lysate of a cell infected with the target. In some embodiments, barcode moieties are attached to a pool of molecules or molecular fragments of a lysed or fragmented target, optionally a lysed pathogen, to generate a pool of BLAs with antigens that are not predetermined. In some embodiments, barcode moieties are attached to a plurality of molecules or molecular fragments of a lysate of a cell infected with an intracellular pathogen, to generate a pool of BLAs with antigens that are not predetermined. In some embodiments, BLAs comprising a portion of a pathogen that is not predetermined may be used to identify and characterize antibodies against said pathogen, which may be a poorly characterized or newly discovered pathogen.

### Barcode Moieties

As used in the disclosure, “barcode” or “barcode moiety” may refer to an identifiable and/or quantifiable tag. In some embodiments, the barcode moiety is referred to as an antibody identification sequence (AIS). In some embodiments, the barcode moiety of a BLA comprises an oligonucleotide, a peptide, a nano-structure, a fluorescent bead, an isobaric mass tag, or a combination thereof. In some embodiments, the barcode moiety is attachable to the antigen moiety of the BLA without substantial interference with binding of the antigen moiety of the BLA to binding members in the test sample.

In some embodiments, each BLA of a different species comprises a different barcode moiety. In some embodiments, the antigen of each BLA, the species of each BLA, the binding member that binds to the antigen of each BLA, and/or the target is identifiable by the linked barcode moiety. As used in the disclosure, BLAs of the same species may refer to BLAs having barcodes that are identical. As used in the disclosure, barcodes, antigens, BLAs, and binding members may be considered as part of the same species if they can form the same BLA/binding member complexes.

In certain embodiments related to pools of BLAs comprising different antigen moieties, each barcode is specific for one antigen moiety. In other embodiments, each barcode is specific for one target. For example, a pool of BLAs may comprises BLAs comprising different antigenic regions of the same target, wherein each of the BLAs comprise the same barcode, thus allowing the identification of a target based on binding of binding members to different antigenic regions of the target. In other embodiments, each antigenic region of the same target may be associated with a different barcode in the BLA, thus allowing identification and/or quantification of specific antigenic regions of a target bound by the binding members. In certain embodiments, it is contemplated that a pool of BLAs comprises multiple different BLAs comprising various antigenic regions of a first target coupled to the same first barcode, and multiple different BLAs comprising various antigen regions of a second target coupled to the same second barcode, thus allowing for the separate identification and/or quantification of binding members that bind to either the first target or the second target. A pool of BLAs may further comprise additional different BLAs that comprise various antigenic regions of third, fourth, fifth or more targets, wherein BLAs comprising antigenic regions of the same targets share the same barcode.

In some embodiments, each barcode moiety comprises an oligonucleotide, wherein each oligonucleotide comprises a unique sequence, one or more primer binding sites, and, optionally, a cleavage site. In some embodiments, each different BLA is identifiable by its unique oligonucleotide sequence. In some embodiments, the target from which the antigen of each BLA was derived is identifiable by the unique sequence.

In some embodiments, each oligonucleotide is about the same length. In some embodiments, the base composition of each oligonucleotide is substantially the same as any other oligonucleotide. Having oligonucleotides of about the same length and substantially the same base composition may avoid potential bias in amplification and purification efficiency. Such bias, if present, may shift the relative abundance of different species of BLAs prior to detection and quantification. For example, a shorter oligonucleotide linked to antigen A may amplify more efficiently than a longer oligonucleotide linked to antigen B during PCR, increasing its prevalence in the resulting population of amplicons, thereby creating a bias that overrepresents the shorter oligonucleotide. This may result in quantification results with falsely high amounts of binding members directed to antigen A in the test sample.

In some embodiments, the unique sequence of each oligonucleotide is generated by a computational method. In some embodiments, the unique sequence of each oligonucleotide is selected from a predetermined pool of unique sequences. In some embodiments, each unique sequence is discrete from any other unique sequence of the pool of unique sequences by Hamming distance of at least 2. In some embodiments, a unique sequence is assigned to each different antigen.

In some embodiments, the BLA is constructed such that the oligonucleotide can be separated from the antigen, e.g., so as to isolate the oligonucleotide portion of the bound BLAs before further analysis, such as, e.g., sequencing. In some embodiments, the oligonucleotide comprises a cleavage site and can be separated from the antigen by treatment with one or more restriction enzymes. In some embodiments, the cleavage site of each oligonucleotide comprises a restriction site for an endonuclease. In some embodiments multiple BLAs may be present with different sites to facilitate cleavage by different restriction enzymes. In some embodiments, the cleavage site of each oligonucleotide is located between the linkage to the antigen and the primer binding site. In some embodiments, the restriction site is identical for all BLAs. Illustrative examples of said restriction enzymes may include, but are not limited to: Aft II, AgeI, BamHI, BspE I, Dra I, EcoR V, Hind III, Nco I, Pst I, Sma I, BsuRI, Hae III, and Xba I. In some embodiments, the restriction enzyme is BsuRI or HaeIII.

In some embodiments, the oligonucleotide comprises one or more primer binding sites, e.g., to facilitate amplification and/or sequencing. In some embodiments, the oligonucleotide comprises two primer binding sites. In some embodiments, the primer binding sites comprise different sequences. In some embodiments, the primer binding sites comprise the same sequence. In some embodiments, each primer binding site comprises a sequence complementary to an amplification primer. In some embodiments, the primer binding sites comprise SEQ ID NO: 4 and SEQ ID NO: 5.

In some embodiments, the amplification primer comprises an adaptor sequence compatible with a sequencing method and a sequence complementary to the primer binding site of the oligonucleotide. In some embodiments, the amplification primers comprise SEQ ID NO: 8 and SEQ ID NO: 9. The adaptor sequence may be complementary to the chemistry used to secure DNA to a surface during a sequencing process. In some embodiments, the adaptor sequence is a sequence that allows binding to a sequencer flow cell. In some embodiments, the adaptor sequence is an Illumina P5/P7 sequence. In some embodiments, the adaptor sequences comprise SEQ ID NO: 10 and SEQ ID NO: 11. In some embodiments, the amplification primer comprises a sequencing primer binding site. In some embodiments, the sequencing primer binding site comprises a nucleotide sequence corresponding to SEQ ID NO: 12.

In some embodiments, the compositions of the disclosure comprise a pool of BLAs, wherein each BLA comprises an antigen linked to a barcode moiety comprising an oligonucleotide. In some embodiments, each oligonucleotide of a plurality of oligonucleotides comprises a unique sequence, one or more primer binding sites, and a cleavage site, wherein the antigen of each BLA is identifiable by the unique sequence, wherein the target from which the antigen of each BLA was derived is identifiable by the unique sequence, wherein each oligonucleotide is about the same length, wherein the base composition of each oligonucleotide is substantially the same as any other oligonucleotide, wherein the unique sequence of each oligonucleotide is generated by a computational method, wherein each unique sequence is discrete from any other unique sequence of the pool of unique sequences by Hamming distance of at least 2, wherein a unique sequence is assigned to each different antigen, and wherein the cleavage site of each oligonucleotide comprises a restriction site for an endonuclease.

### Linkage of Antigen and Barcode

Within the context of a BLA, the barcode is linked to the antigen. As used in the disclosure, “link” may refer to a physical linkage or a general association. For example, a “linked” barcode of an antigen may be a physically attached barcode moiety of said antigen, or a separated barcode moiety that was previously attached to said antigen, or a barcode that was associated/paired with said antigen (e.g., through computational methods). In some embodiments, the barcode moiety can be separated from its linked antigen.

In some embodiments, each antigen is attached to its linked barcode moiety by a bond or linker. In some embodiments, each antigen is attached to its linked barcode moiety by a reagent capable of conjugating an oligonucleotide to a polypeptide. In some embodiments, the bond or linker comprises a heterobifunctional linker. In some embodiments, the bond or linker comprises HyNic-4FB, Succinimidyl 6-hydrazinonicotinate acetone hydrazone (SANH), succinimidyl 4-(N-maleimidomethyl) cyclohexane-1-carboxylate (SMCC), or biotin-streptavidin. In some embodiments, the bond or linker is cleavable or breakable.

### Illustrative Methods

The methods disclosed herein may be practiced using various BLAs comprising various barcodes and antigens, to identify and/or quantitate a variety of different binding members from a variety of different types of test samples, including but not limited to those specifically disclosed herein. Illustrative methods for producing BLAs, preparing compositions for use in the disclosed assays, and methods of conducting the disclosed assays are disclosed.

### Generating BLAs and BLA Pools

BLA species may be synthesized as disclosed herein. For example, specific barcodes may be linked to particular antigens as described above. After synthesis, the concentration and volume of each BLA species may be determined by determining the concentration of the linked antigens. For example, in some embodiments where the antigen is a protein, BLA concentrations may be determined using any known method of protein concentration measurement, including, but not limited to, Bradford assay and BCA (Bicinchoninic Acid) assay. In some embodiments, BLA concentration is determined by Bradford assay.

In some embodiments, each synthesized BLA species is combined with other BLA species to generate a pool of different BLA species. In some embodiments, each BLA of the pool of BLAs is present at a known concentration. In some embodiments, each BLA of the pool of BLAs is present at the same or similar concentrations, which may be based on the concentration of the BLA species having the lowest determined concentration. In some embodiments, the ratio of each BLA relative to every other BLA is predetermined and/or optimized through testing.

In some embodiments, the concentration of each BLA species in the pool of BLAs is not determined.

In some embodiments, the pool of BLAs comprise different antigens, where at least two different antigens are from the same target. In some embodiments, the pool of BLAs comprise different antigens, where the different antigens are from at least two different targets.

In some embodiments, BLAs comprising different antigens comprise different barcode moieties. For example, in some embodiments, in a pool of BLAs comprising different species of BLAs, each antigen of a different species is linked to a different barcode moiety.

In some embodiments, BLAs comprising different antigens from the same target comprise identical barcode moieties. For example, in some embodiments, in a pool of BLAs comprising different species of BLAs, all antigens from the same virus (target) are linked to the same barcode moiety.

The compositions and methods of the disclosure may be used to generate a BLA pool comprising more than 1, more than 2, more than 5, more than 10, more than 50, more than 100, more than 200, more than 500, or more than 1000 different BLAs. The compositions and methods of the disclosure may be used to generate a BLA pool comprising more than 1, more than 2, more than 5, more than 10, more than 50, more than 100, more than 200, more than 500, or more than 1000 different BLAs.

In some embodiments, during the assay, when the test sample is combined with the BLAs, the amount of each BLA present is significantly higher than the amount of its corresponding binding member in the test sample. The amount of each BLA may be about 2×, about 3×, about 4×, about 5×, about 6×, about 7×, about 8×, about 9×, about 10×, or more than 10× higher than its corresponding binding member in the test sample. The amount may refer to concentration in a composition comprising the BLAs and the test sample, or the amount may refer to the molar amount of the BLA and the binding member present in the composition comprising the BLAs and test sample.

In some embodiments, the amount of each BLA is not significantly higher than the amount of its corresponding binding member in the test sample. In some embodiments, the positive pool and negative pool described in the disclosure may be sufficient to quantify binding members in the test sample, without using an excessive amount of BLA species.

Mixing a Test Sample with a Pool of BLAs

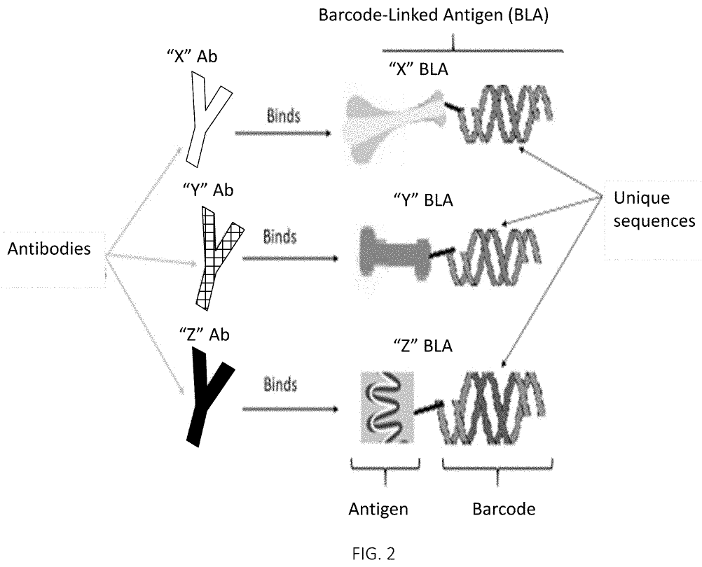

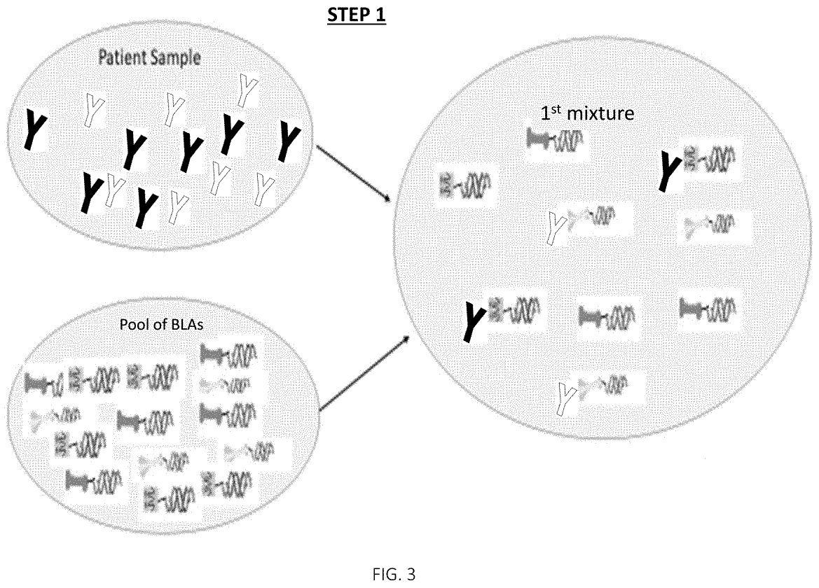

In some embodiments, a test sample comprising different binding members, e.g., antibodies, is mixed with a pool of BLAs (starting BLA pool). The test sample may be a biological sample obtained from a subject, such as a blood or serum sample obtained from a patient. As depicted in FIG. 2, each different BLA in FIG. 2 is represented by a different letter (X, Y, and Z). Similarly, each different antibody in FIG. 2 is represented by a different letter (X, Y, and Z), wherein each antibody binds to the antigen of each corresponding BLA. As depicted in the embodiment of FIG. 3, a first mixture is formed by mixing the patient sample comprising “X” and “Z” antibodies with a pool of BLAs comprising “X”, “Z”, and “Y” BLAs. In this example, the patient lacks “Y” antibodies, thus the “Y” BLAs are not bound to antibodies. In some embodiments, BLAs will be in excess of antibodies in the sample, such that substantially all antigen-specific antibodies in the test sample will bind to the corresponding antigens of the BLAs and form BLA/antibody complexes. In the embodiment depicted in FIG. 3, the resulting first mixture comprises “X” and “Z” BLA/patient antibody complexes, “Y” unbound BLAs (due to lack of “Y” antibodies in the test sample), and excess “X” and “Z” unbound BLAs.

In some embodiments, a negative sample control that does not comprise any binding members is contacted with a starting BLA pool. The negative sample control may be a biological sample that does not comprise antibodies, such as antibody-depleted serum.

In some embodiments, a positive sample control that is known to comprise binding members is contacted with a starting BLA pool.

### Labeling BLA/Antibody Complexes

In some embodiments, the BLA/binding members are labelled with a label agent capable of binding to the BLA/binding member complex and capable of being isolated from mixture. In some embodiments, the label agent binds to the binding member. In some embodiments, the label agent is capable of attaching to a solid support. The solid support may be selected from the group consisting of: a solid matrix, a protein-coated solid matrix, a cell-coated solid matrix, free-floating particles, a column of particles, labeled beads, magnetic beads, non-magnetic beads, protein-coated magnetic beads, a slurry of particles suspended in liquid, a slurry of protein-coated particles suspended in liquid, and a flow-through column. In some embodiments, the solid support is magnetic.

In some embodiments, said label agent is a biotin-labeled anti-human antibody. The biotin-labeled anti-human antibody may be selected from many commercially available anti-human antibodies, including, but not limited to, goat, rabbit, mouse, chicken anti-human antibodies. In some embodiments, the anti-human antibody is an Fc specific IgG produced in goat.

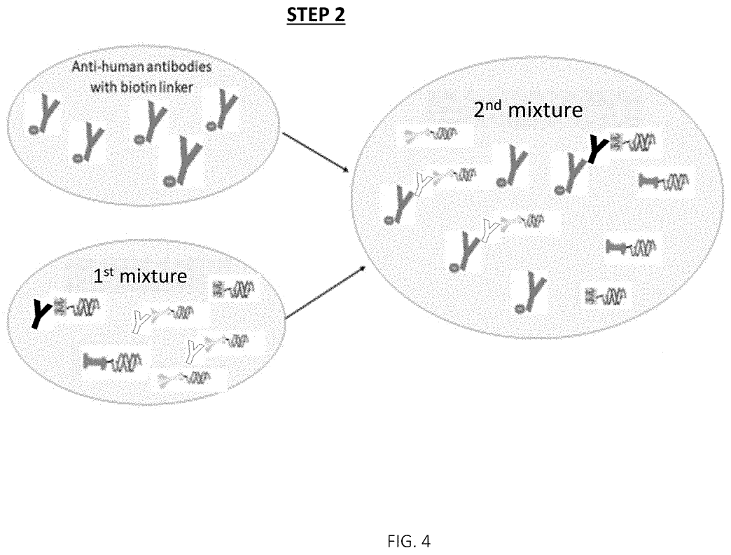

As depicted in FIG. 4, in some embodiments, anti-human antibodies having a biotin linker are added to the first mixture to form a second mixture. In some embodiments, the anti-human antibodies are in excess of the antibodies in the first mixture, such that substantially all antibodies of the first mixture will bind to the anti-human antibodies. As depicted in FIG. 4, the resulting second mixture comprises “X” and “Z” BLA/patient antibody/anti-human antibody complexes, unbound anti-human antibodies, “Y” unbound BLAs, and excess “X” and “Z” unbound BLAs.

### Isolating Labeled BLA/Antibody Complexes

In some embodiments, the methods of the disclosure further comprise isolating the labeled BLA/binding member complexes from the mixture. In some embodiments, the isolated labeled BLA/binding member complexes are also referred to as captured complexes.

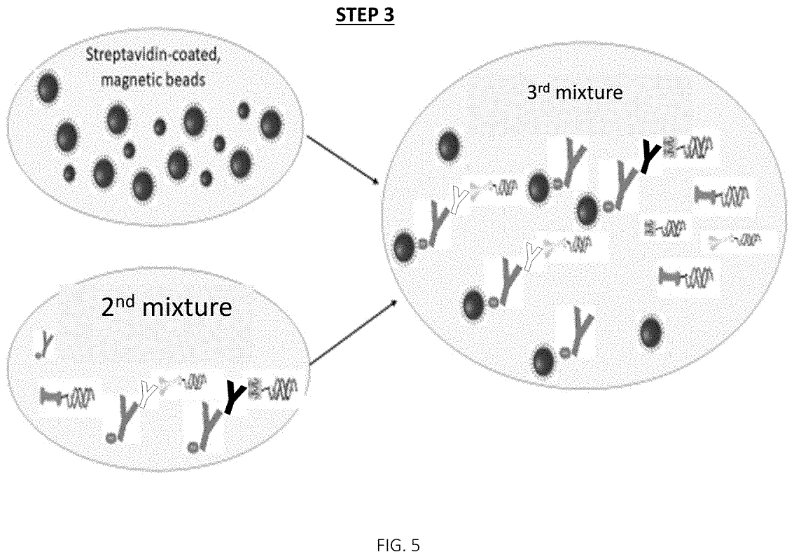

As depicted in FIG. 5, in some embodiments, streptavidin-coated magnetic beads are added to the second mixture to form a third mixture. In some embodiments, the streptavidin-coated magnetic beads are in excess of the biotin-labeled anti-human antibodies, such that substantially all anti-human antibodies of the second mixture will bind to the beads. The resulting third mixture as depicted in FIG. 5 comprises “X” and “Z” BLA/patient antibody/anti-human antibody/beads complexes, anti-human antibody/beads complexes, excess unbound beads, “Y” unbound BLAs, and excess “X” and “Z” unbound BLAs.

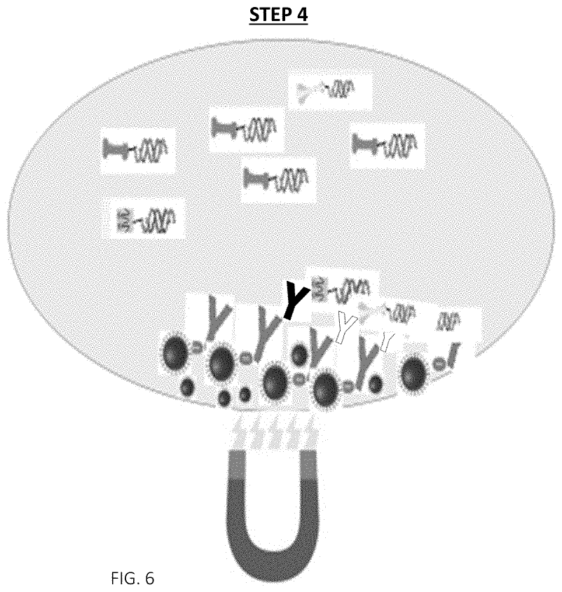

In some embodiments, a magnetic force is applied to the third mixture to separate the BLA/patient antibody/anti-human antibody/beads complexes from the BLAs that are not bound to antibodies, as depicted in FIG. 6. In this example, BLAs that are not bound to their corresponding antibodies (e.g. “Y” unbound BLAs, excess “X” BLAs, and excess “Z” BLAs) are not bound to anti-human antibodies and therefore not bound to magnetic beads, thus these unbound BLAs will not be attracted to the magnetic force and remain suspended in solution.

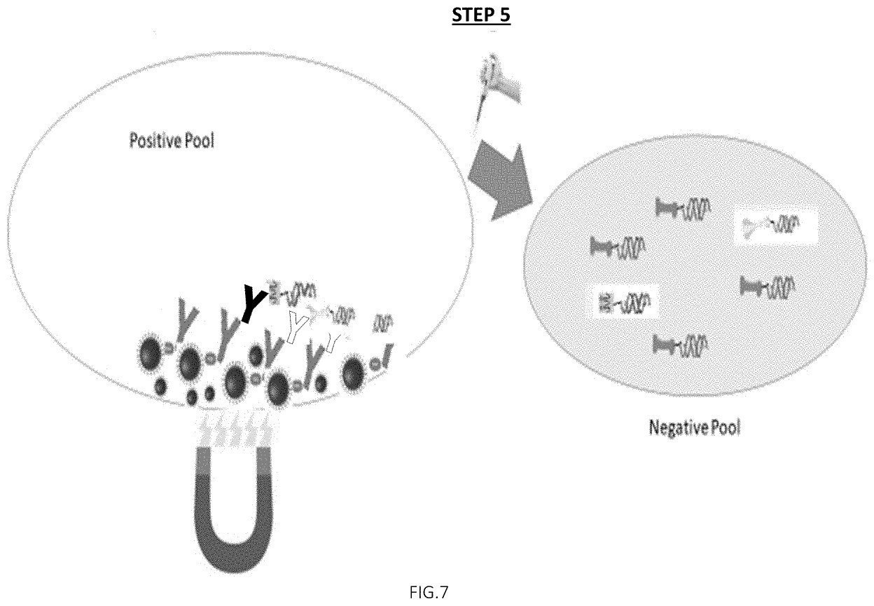

In some embodiments, the BLAs that are not bound to antibodies are removed from the mixture to generate a “negative pool”. As depicted in FIG. 7, the negative pool comprises “Y” unbound BLAs and the excess “X” and “Z” unbound BLAs. Simultaneously, a “positive pool” is generated, where the positive pool comprises “X” and “Z” BLA/patient antibody/anti-human antibody/beads complexes, anti-human antibody/beads complexes, and excess unbound beads. Upon removal of the negative pool, the positive pool may be resuspended in liquid and removed from the magnet. In some embodiments, the positive pool is analyzed to test for the presence of patient antibodies, and the negative pool may be analyzed as a control.

In some embodiments, BLA barcode moieties for binding members that the test sample lack will only be present in the negative pool, while barcode moieties for binding members in the test sample will be present in the positive pool, and to a lesser degree, the negative pool (i.e., the BLAs that were in excess of their corresponding binding members). By comparing amount of barcode moieties in the positive pool to the amount in the negative pool, the quantity of binding members in the test sample may be determined. Specifically, decreased BLAs in the negative pool as compared to the original BLA pool (i.e., the composition added to the test sample) indicates the presence of corresponding binding members in the test sample, and the level of this decrease can be used to quantify the amount of the corresponding binding member in the test sample. The analysis of the negative pool may be done independently from the analysis of the positive pool, or to supplement the quantification of binding members in the positive pool.

In some embodiments, the negative pool is generated but not analyzed. In some embodiments, the negative pool is disposed after separation from the positive pool.

Separating the Barcode from the Antigen of Each BLA

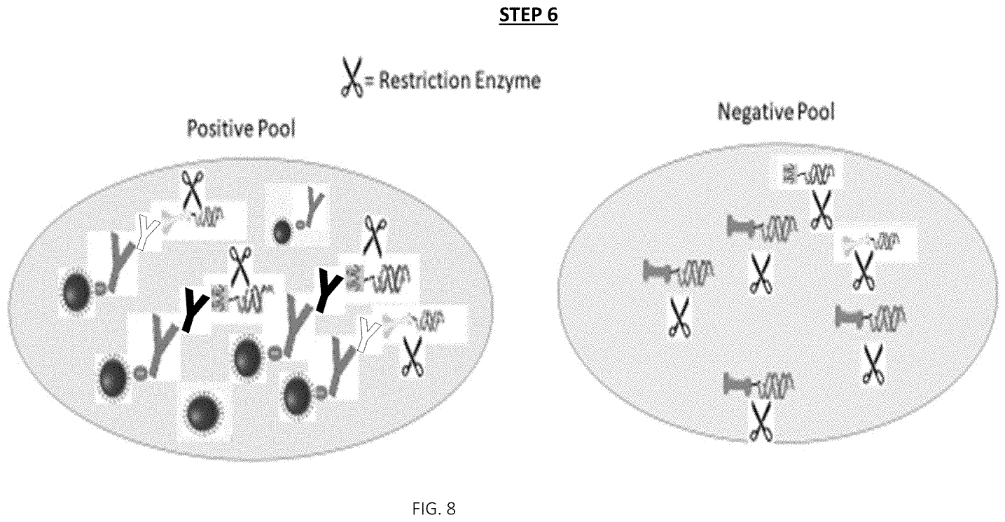

In some embodiments, one or more restriction enzymes are added to the positive pool, the negative pool, or both. In some embodiments, as depicted in FIG. 8, the restriction enzyme targets a cleavage site in the barcode, thus separating the barcode oligonucleotide from the antigen. In some embodiments, the separated barcodes are also referred to as captured barcodes.

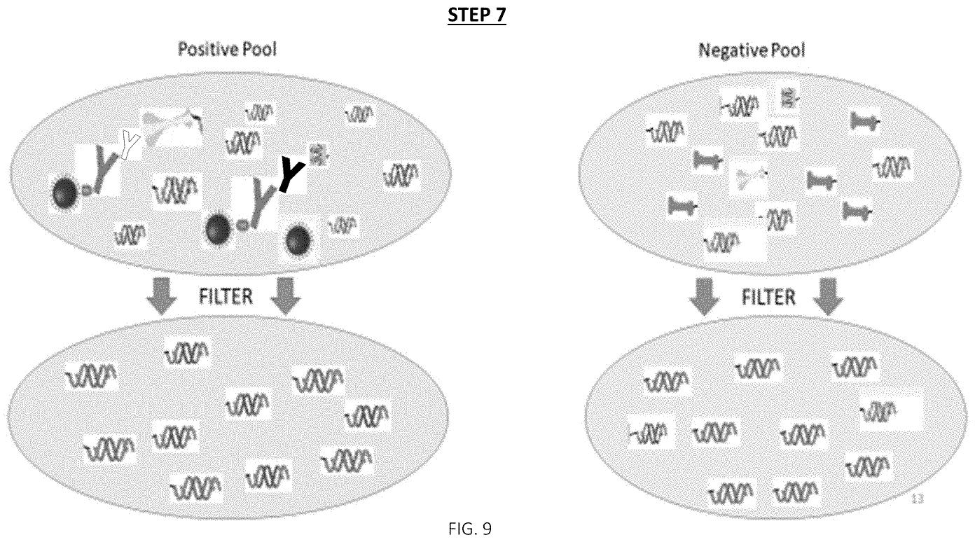

In some embodiments, the separated barcodes are purified from the mixture. In some embodiments, purification is achieved by filtration, as depicted in FIG. 9. The resulting purified positive and/or negative pools comprise separated barcode oligonucleotides. The purified positive pool may comprise barcodes identifying all the binding members present in the test sample that were capable of to form BLA/antibody/bead complexes.

The level of each barcode species in the purified positive pool relative to every other barcode species may be proportional to the level of the corresponding binding member relative to the other binding members in the test sample. The purified negative pool may comprise the barcodes identifying all the BLAs that the test sample didn't have corresponding binding members for. For the negative pool, the changes in the amounts of barcodes relative to the starting BLA pool (i.e. the BLA pool added to the test sample) are proportional to the binding member amounts in the test sample.

### Amplifying the Separated Barcodes

In some embodiments, the methods of the disclosure preserve the quantitative makeup of the positive and/or negative pool such that the prevalence of each barcode in the positive and/or negative pool reflects the prevalence of the binding member in the test sample. In some embodiments, the barcodes are designed to preserve the quantitative makeup of the pool through PCR amplification and sequencing by minimizing bias caused by barcode size differences or sequence variations.

In some embodiments, the separated and/or separated and purified barcode oligonucleotides are amplified by PCR to create amplified barcodes compatible for sequencing. In some embodiments, amplification may be performed directly on the BLA/binding member complexes without first separating the barcode moiety from the antigen.

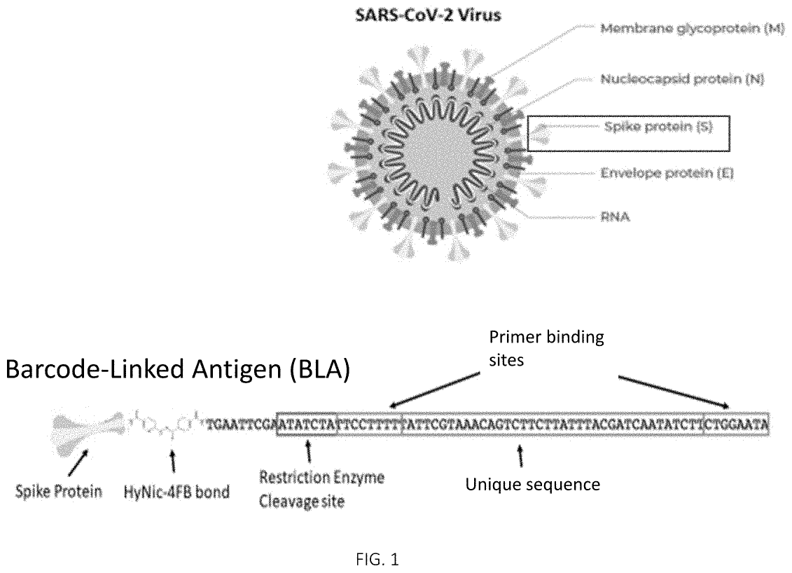

In some embodiments, the barcodes used for amplification each comprise two primer binding sites, as depicted in FIG. 1. In some embodiments, the primers used in PCR each comprise a sequence complementary to the primer binding site of each barcode and an adaptor sequence compatible with a sequencing method. In some embodiments, the adaptor sequence is a sequence that allows binding to a sequencer flow cell. In some embodiments, the adaptor sequence is an Illumina P5/P7 sequence.

In some embodiments, during amplification the PCR primers are incorporated in their entirety into each new amplicon, thus the adaptor sequences are also incorporated into the amplicons. In some embodiments, at the end of a single amplification step, each of the resulting amplicons comprises, from 5′ to 3′, a first adaptor sequence, a first primer binding site, a unique sequence, a second primer binding site, and a second adaptor sequence.

### Quantifying the Amplified Barcodes

In some embodiments, the amplified barcodes are sequenced and analyzed. In some embodiments, the sequence of each unique barcode indicates the identity of its originally linked antigen, which indicates the identity of the binding member bound to this antigen in the test sample. This identification may provide qualitative testing for binding members present in the test sample. In some embodiments, the quantity of each unique barcode indicates the quantity of its originally linked antigen, which indicates the quantity of the binding member bound to this antigen in the test sample. This quantification may provide quantitative testing for binding members present in the test sample.

In some embodiments, sequencing by synthesis is used to identify and quantify the barcodes within each pool. The barcodes may include adapter sequences flanking either side of the unique sequence region. These adapter sequences may be specific to the manufacturer of the particular model of DNA sequencer employed and facilitate the binding of barcode oligonucleotides to the flow cell. During the sequencing process the barcode oligonucleotides may be distributed across a flow cell containing millions of individual wells such that each well will contain a single barcode oligonucleotide. Sequencing of the barcode oligonucleotides may proceed with the instrument monitoring and detecting each well separately. At the end of the sequencing run, the sequence of each barcode oligonucleotide loaded onto the flow cell may be analyzed and sorted into groups to identify all the different barcodes and how many of each are present. This technique is able to sample millions of individual barcodes, thus providing a wide dynamic range allowing the accurate quantification of rare occurrences as well as frequent ones.

In some embodiments, each amplified positive and/or negative pool contains multiple barcode species. Each barcode species may comprise a unique sequence that is common to all members of the species. Each barcode species may correspond to a specific binding member species that binds its linked antigen. In some embodiments, the barcode species present in the positive pool will correspond to binding members that were present in the test sample, while the number of said barcode species is proportional to the number of binding members present in the test sample.

In some embodiments, the barcodes in the negative pool are from BLAs that remained unbound after being exposed to the binding members of the test sample. In some embodiments, the negative pool can provide information about the identity and quantity of binding members in the test sample by comparing the number of barcodes in the negative pool to the number of barcodes in the starting BLA pool before it was exposed to the test sample. Barcode species that are less prevalent in the negative pool when compared to the unexposed starting BLA pool may be the result of the binding members of that species pulling their corresponding BLAs into the positive pool. In some embodiments, the difference between the amount of a barcode species in the negative pool compared to its amount in the starting BLA pool can be used to infer the number of binding members that were present in the test sample, where a large difference between the negative pool and the starting BLA pool indicates the test sample contains a large number of binding members and a small difference indicates the test sample contains a small number of binding members. A test sample lacking binding members of a certain species may produce a negative pool where the number of barcodes of said species remains unchanged compared to its number in the starting BLA pool.

In some embodiments, a quantitative relationship between the number of binding members of a certain species in the test sample and the number of barcodes of the same species in the positive pool exists for each species of BLA/binding member complex for which there is a BLA included in the starting BLA pool. For example, contacting a test sample having a high number of a antibodies with a BLA pool of the disclosure using methods of the disclosure may produce a positive pool with a corresponding high number of a barcodes; contacting a test sample having a low number of β antibodies with a BLA pool of the disclosure using methods of the disclosure may produce a positive pool with a low number of β barcodes; contacting a test sample that does not contain any γ antibodies with a BLA pool of the disclosure using methods of the disclosure may generate a positive pool that does not contain any γ barcodes. Thus, the identity and prevalence of different species of antibodies in each test sample may be determined by identifying and counting the barcodes present in each corresponding positive pool.

In some embodiments, the quantitative relationship between binding members in the test sample and barcodes in the positive pool is consistent within barcode/binding member pairs as well as between different barcode/binding member pairs, such that if a test sample contains three times as many α antibodies as β antibodies, the positive pool generated by the sample would contain more a barcodes relative to β barcodes than a pool generated from a test sample that contains the same number of α and β antibodies. In some embodiments, the quantitative relationship between binding members in the test sample and barcodes in the positive pool is substantially linear. In some embodiments, the slope of said linear relationship is about 1, for example, a three-fold increase in α antibodies in a test sample would lead to exactly to a three-fold increase in α barcodes in the positive pool generated. In some embodiments, the slope of said linear relationship is not 1, for example, a threefold increase in a antibodies may lead to a two-fold or four-fold increase in α barcodes in the positive pool generated. Different antibodies may have different efficiencies in translating antibody levels into barcode levels, but regardless of the individual behavior of antibodies, the methods of the disclosure allow quantification of antibody levels based on quantification of barcodes because a substantially linear relationship can be established between the two.

In some embodiments, further information can be provided by comparing the levels of different species of binding members within the same sample. In some embodiments, the absolute number measured for a binding member of interest is compared to the absolute number of a “control” binding member species. For example, certain antibodies are known to be present in most patient samples at a substantially consistent level, thus they can be used as an internal control to gauge whether the antibody species of interest is present at high or low levels. In some embodiments, more than one control species of antibody may be used to set a baseline for determining the levels of the antibodies of interest.

### Kits

In some embodiments, the disclosure provides kits that may be used to quantify binding members in a test sample, where the kit comprises a first vessel comprising a composition comprising at least one BLA, wherein each BLA comprises a barcode moiety attached to an antigen of a target, including any of the various embodiments of BLAs disclosed herein. In some embodiments of the kit of the disclosure, at least one target is a pathogen or infectious agent. In some embodiments of the kit of the disclosure, the pathogen is a virus, a bacterium, a fungi, a protozoa, a worm, or a prion. In some embodiments of the kit of the disclosure, the virus is a SARS-CoV-2 virus. In some embodiments of the kit of the disclosure, the test sample is a biological sample obtained from a subject. In some embodiments of the kit of the disclosure, the subject is a human. In some embodiments of the kit of the disclosure, the biological sample comprises serum, plasma, whole blood, saliva, mucus, or a combination thereof. In some embodiments of the kit of the disclosure, the binding members quantified by the kit comprise antibodies.

In some embodiments, the first vessel of the kit comprises a pool of BLAs. The pool of BLAs may all be identical. In certain embodiments, the pool of BLAs may comprise BLAs comprising different antigens. In some embodiments of the kit of the disclosure, at least two different antigens are from the same target. In some embodiments of the kit of the disclosure, BLAs of the kit comprising different antigens from the same target comprise identical barcode moieties. In some embodiments of the kit of the disclosure, the different antigens are from at least two different targets. In some embodiments of the kit of the disclosure, BLAs of the kit comprising different antigens comprise different barcode moieties. In some embodiments of the kit of the disclosure, at least one antigen of the kit comprises a polypeptide, or an immunogenic fragment, variant, or epitope thereof. In some embodiments of the kit of the disclosure, at least one antigen comprises a polysaccharide.

In some embodiments, the kit further comprises a second vessel comprising a reagent capable of separating the antigen from the attached barcode moiety of each BLA. In some embodiments of the kit of the disclosure, said reagent comprises a restriction enzyme.

In some embodiments of the kit of the disclosure, each antigen is attached to its linked barcode moiety by a bond or linker. The bond or linker may comprise a heterobifunctional linker. In some embodiments of the kit of the disclosure, the bond or linker comprises HyNic-4FB, Succinimidyl 6-hydrazinonicotinate acetone hydrazone (SANH), succinimidyl 4-(N-maleimidomethyl) cyclohexane-1-carboxylate (SMCC), or biotin-streptavidin. In some embodiments of the kit of the disclosure, the bond or linker is cleavable or breakable.

In some embodiments of the kit of the disclosure, each barcode moiety comprises an oligonucleotide, a peptide, a nano-structure, a fluorescent bead, an isobaric mass tag, or a combination thereof.

In some embodiments, the kit further comprises a third vessel comprising a labelled agent capable of binding to the binding members. In some embodiments, the third vessel comprises labelled anti-human antibodies. In some embodiments of the kit of the disclosure, the label is capable of attaching to a solid support. In some embodiments of the kit of the disclosure, the label is a biotin label and the solid support is magnetic. In some embodiments, the kit further comprises a fourth vessel comprising the solid support.

In some embodiments of the kit of the disclosure, each barcode moiety comprises an oligonucleotide. In some embodiments of the kit of the disclosure, each oligonucleotide comprises a unique sequence, one or more primer binding sites, and, optionally, a cleavage site. In some embodiments of the kit of the disclosure, the antigen of each BLA is identifiable by the unique sequence. In some embodiments of the kit of the disclosure, the target from which the antigen of each BLA was derived is identifiable by the unique sequence. In some embodiments of the kit of the disclosure, each primer binding site comprises a sequence complementary to an amplification primer, wherein the amplification primer comprises an adaptor sequence compatible with a sequencing method.

In some embodiments of the kit of the disclosure, each barcode oligonucleotide is about the same length. In some embodiments of the kit of the disclosure, the base composition of each barcode oligonucleotide is substantially the same as any other oligonucleotide. In some embodiments of the kit of the disclosure, the unique sequence of each barcode oligonucleotide is generated by a computational method. In some embodiments of the kit of the disclosure, the unique sequence of each barcode oligonucleotide is selected from a predetermined pool of unique sequences. In some embodiments of the kit of the disclosure, each unique sequence is discrete from any other unique sequence of the pool of unique sequences by Hamming distance of at least 2. In some embodiments of the kit of the disclosure, a unique sequence is assigned to each different antigen. In some embodiments of the kit of the disclosure, the cleavage site of each barcode oligonucleotide comprises a restriction site for an endonuclease.

In some embodiments, the kit further comprises a fifth vessel comprising an amplification primer.

In some embodiments, the kit further comprises a sixth vessel comprising a negative sample control that does not comprise binding members.

In some embodiments, the kit further comprises a seventh vessel comprising a positive sample control comprising binding members.

In some embodiments, the kit may further comprise one or more vessels comprising one or more serum calibrators. In some embodiments of the kit of the disclosure, the serum calibrator may comprise serum or serum-mimic. The calibrating serum or serum-mimic may be used to gauge test results, in which the test results are normalized against the serum or serum-mimic.

In some embodiments of the kit of the disclosure, the vessels may be made of glass or a plastic to which the recited reagents adhere poorly. In some embodiments, the vessels may comprise one or more coatings to decrease adherence of reagents, such as polyethylene glycol (PEG) coatings and coatings of polytetrafluoroethylene (PTFE).

In certain embodiments of the kit of the disclosure, each of the vessels may contain an amount of the recited ingredient to carry out at least one test.

In some embodiments of the kit of the disclosure, the instructions for carrying out a test may also be present in the kit. A contemplated kit may be provided as a container that holds the recited components.

In some embodiments, the kit of the disclosure may further include other components used m preparing and performing the methods of the disclosure. Other contents of the kit may comprise:
Packing materials
Phosphate buffered saline (PBS)
dNTPs
Polymerases
PCR master mixes
Dimethyl sulfoxide (DMSO)
serological pipettes
1.5 mL centrifuge tubes
50 mL conical tubes

### Additional Definitions

Unless otherwise defined in the disclosure, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described in the disclosure, are those well-known and commonly used in the art.

As used in the disclosure, the following terms have the meanings ascribed to them unless specified otherwise.

The articles “a,” “an,” and “the” are used in the disclosure to refer to one or to more than one (i.e., to at least one) of the grammatical object of the article. By way of example, “an element” means one element or more than one element.

The use of the alternative (e.g., “or”) should be understood to mean either one, both, or any combination thereof of the alternatives.

The term “and/or” should be understood to mean either one, or both of the alternatives.

As used in the disclosure, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

Throughout this specification, unless the context requires otherwise, the words “comprise”, “comprises,” and “comprising” will be understood to imply the inclusion of a stated step or element or group of steps or elements but not the exclusion of any other step or element or group of steps or elements. In some embodiments, the terms “include,” “has,” “contains,” and “comprise” are used synonymously.

By “consisting of” is meant including, and limited to, whatever follows the phrase “consisting of” Thus, the phrase “consisting of” indicates that the listed elements are required or mandatory, and that no other elements may be present.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the disclosure. Thus, the appearances of the foregoing phrases in various places throughout this specification are not necessarily all referring to the same embodiment. Furthermore, the particular features, structures, or characteristics may be combined in any suitable manner in one or more embodiments.

As used in the disclosure, the term “isolated” means material that is substantially or essentially free from components that normally accompany it in its native state. In some embodiments, the term “obtained” or “derived” is used synonymously with isolated.

A “subject,” “individual,” or “patient” as used in the disclosure, includes any animal that exhibits a symptom of a condition that can be detected or identified with compositions of the disclosure. Suitable subjects include laboratory animals (such as mouse, rat, rabbit, or guinea pig), farm animals (such as horses, cows, sheep, pigs), and domestic animals or pets (such as a cat or dog). In some embodiments, the subject is a mammal. In certain embodiments, the subject is a non-human primate, and, in preferred embodiments, the subject is a human.

### EQUIVALENTS

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

Furthermore, it is intended that any method described in the disclosure may be rewritten into Swiss-type format for the use of any agent described in the disclosure, for the manufacture of a medicament, in treating any of the disorders described in the disclosure. Likewise, it is intended for any method described in the disclosure to be rewritten as a compound for use claim, or as a use of a compound claim.

All publications, patents, and patent applications described in the disclosure are hereby incorporated by reference in their entireties.

### EXAMPLES

The disclosure is further illustrated by the following examples, which are not to be construed as limiting this disclosure in scope or spirit to the specific procedures described in the disclosure. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure.

### Example 1—Preparation of Blas and Antibody Samples

To evaluate the performance of BLAs in quantification of antibodies in a sample, 12 samples with known antibody levels were measured using BLAs comprising spike protein antigen and nucleocapsid antigen from the SARS CoV-2 virus. The composition of each sample is shown in Table 1.

TABLE 1
Samples Tested

| | | Number of | Number of |
|---|---|---|---|
| | | anti-Spike | anti-Nucleocapsid |
| Sample | | Antibodies | Antibodies |
| # | Sample Name | in Sample | in Sample |
| 1 | Spike 0 Nuc 10,000 | 0 | 10,000 |
| 2 | Spike 100 Nuc 10,000 | 100 | 10,000 |
| 3 | Spike 1,000 Nuc 10,000 | 1,000 | 10,000 |
| 4 | Spike 10,000 Nuc 10,000 | 10,000 | 10,000 |
| 5 | Spike 100,000 Nuc 10,000 | 100,000 | 10,000 |
| 6 | Spike 1,000,000 | 1,000,000 | 10,000 |
| | Nuc 10,000 | | |
| 7 | Spike 10,000 Nuc 0 | 10,000 | 0 |
| 8 | Spike 10,000 Nuc 100 | 10,000 | 100 |
| 9 | Spike 10,000 Nuc 1,000 | 10,000 | 1,000 |
| 10 | Spike 10,000 Nuc 10,000 | 10,000 | 10,000 |
| 11 | Spike 10,000 Nuc 100,000 | 10,000 | 100,000 |
| 12 | Spike 10,000 Nuc | 10,000 | 1,000,000 |
| | 1,000,000 | | |

Samples #1 to #6 each contained 10,000 anti-nucleocapsid antibodies and varying levels of anti-spike antibodies (from 0 to 1,000,000). Samples #7-#12 each contained 10,000 anti-spike antibodies and varying levels of anti-nucleocapsid antibodies (from 0 to 1,000,000). This setup allowed measurement of the linearity of the BLA signal for each antibody in the presence of other antibodies, which mimics the antibodies present in a patient sample.

BLAs used comprise two species: one comprises a spike protein antigen (S-BLA) and the other comprises a nucleocapsid antigen (N-BLA). The spike protein antigen comprises an amino acid sequence shown in SEQ ID NO: 13, and the nucleocapsid antigen comprises an amino acid sequence shown in SEQ ID NO: 14. A schematic diagram showing the structure of the S-BLA used in this example is shown in FIG. 1. The barcode moiety is an oligonucleotide having SEQ ID NO: 1 for S-BLA and SEQ ID NO: 2 for N-BLA. The oligonucleotides of S-BLAs and N-BLAs are linked to their respective antigens by a HyNic-4FB bond. Each oligonucleotide of the S-BLAs and N-BLAs comprises a restriction enzyme cleavage site (SEQ ID NO: 3), two primer binding sites (SEQ ID NO: 4 and SEQ ID NO: 5), and a unique sequence between the primer binding sites. For S-BLAs, the unique sequence is SEQ ID NO: 6; for N-BLAs, the unique sequence is SEQ ID NO: 7.

BLAs were synthesized under a modified protocol using the SoluLINK® Protein Oligo Conjugation Kit (Cat. No. S-9011-1). The proteins and oligos used for conjugation were first selected/conformed to meet the following specifications:

### Proteins:

Protein molecular weight range 25,000-900,000 Daltons

Protein concentration range 1.0-5.0 mg/ml

Mass of protein 50-650 μg

Protein reaction volume range 50-130 μL

Protein is completely de-salted

### Oligos:

Oligonucleotide size range 20-100 bases

Oligonucleotide concentration range 0.3-0.6 OD₂₆₀/μL

Mass of oligo 15-40 OD₂₆₀

Oligo reaction volume range 30-60 μL

Oligo is completely de-salted

Oligos were rehydrated in TE buffer and concentration was measured by a NanoDrop™ Spectrophotometer (model ND-1000). The oligo solution was desalted using a Zeba Column (Thermo Scientific Cat No. 09882). An S-4FB 2 (succinimidyl 4-formylbenzoate) reagent was added to the desalted oligo solution and incubated at room temperature for 2 hours, after which excess S-4FB was removed. The concentration of the resulting S-4FB-oligo solution was measured by a NanoDrop™ Spectrophotometer at 260 nm according to manufacturer's protocol. The Molar Substitution Ratio (MSR) of the S-4FB-oligo solution was then measured at 360 nm.

1×S-HyNic (Succinimidyl hydrazinium nicotinate) solution was prepared by adding 10 μL of 5.857×S-HyNic solution into 48.57 μL of DMF (1:5.857 dilution). Proteins were desalted and prepared into protein solutions. The appropriate amount of S-HyNic solution was added to the protein solutions and incubated at room temperature for 2.5 hours. The resulting HyNic-modified protein solution was desalted using the Zeba™ Column and equilibrated with Conjugation Buffer.

A conjugation reaction was prepared by adding 2 μL of the HyNic-modified (desalted) protein solution to 18 μL of S-4FB-oligo solution. A control (blank) reaction was prepared by adding 2 μL of Conjugation Buffer to 18 μL of S-4FB-oligo solution. The reactions were mixed well and incubated at 37° C. for 1 hour. After incubation, the reactions were centrifuged to remove condensate. The MSR of the conjugated BLA solutions was measured using a NanoDrop™ Spectrophotometer at 348 nm.

The conjugated BLA solutions were desalted and the final protein concentrations were determined using Bradford assay. The conjugated BLA solutions were purified with a protein A/G column and stored at 4° C.

### Example 2—Generating a Barcode Sequencing Library

The S-BLAs and N-BLAs were pooled into a BLA composition and mixed with each sample of the 12 samples according to methods described in the Exemplary Workflow. Biotin-labeled anti-human antibodies (ThermoFisher Cat. No. 31770) were added to label the BLA/antibody complexes, and magnetic beads (MagnaLink Beads, Vector Lab Cat. No. M-1003-010) were added to form BLA/antibody/anti-human antibody complexes according to methods of the disclosure. BLA/antibody/anti-human antibody/bead complexes were isolated from unbound BLAs using magnetic force, and the unbound BLAs were removed from the mixture. The BLA/antibody/anti-human antibody/bead complexes were then released from the magnet and resuspended in solution.

BsuRI restriction enzymes (Thermo Scientific™ FastDigest BsuRI Cat. No. FD0154) were added to the resuspended BLA/antibody/anti-human antibody/bead complexes, thereby separating the oligonucleotide barcodes from the linked antigens. The separated oligonucleotide barcodes were then purified by filtration using Qiagen Purification Columns (Cat. No. 28104), and the purified oligonucleotides were subject to PCR amplification using amplification primers 1 and 2 (SEQ ID NO: 8 and SEQ ID NO: 9, respectively). After PCR amplification, a sequencing library was generated comprising amplified oligonucleotide barcodes flanked by sequencing adapters on each end. The sequencing adapters (P5/P7) are specific to the sequencer (Illumina MiSeq Cat. No. SY-410-1003) and facilitate the binding of the sequencing library to flow cells of the sequencer. The sequencing adapters used in this example comprise SEQ ID NO: 10 and SEQ ID NO: 11.

### Example 3—Sequencing Analysis and Antibody Quantification

Sequencing by synthesis (Illumina MiSeq Cat. No. SY-410-1003) was used to identify and quantify the amplified oligonucleotide barcodes generated in Example 2.

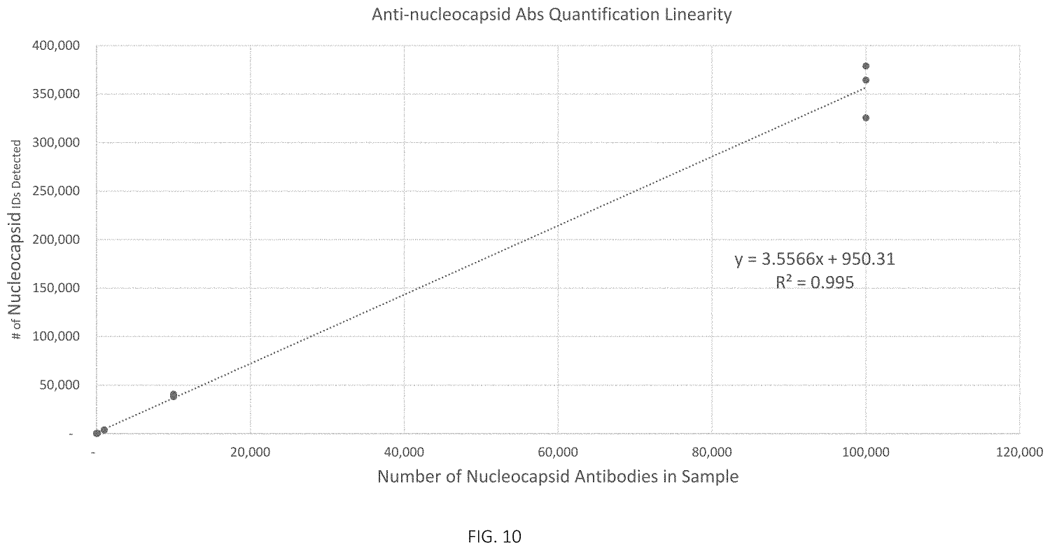

Table 2 shows the detection and quantification of Nucleocapsid barcodes from N-BLAs bound by anti-nucleocapsid antibodies. Results show an extremely linear relationship exists between the number of anti-nucleocapsid antibodies in the sample and the number of nucleocapsid barcodes measured by the sequencer, i.e. as the number of anti-nucleocapsid antibodies increased from sample #7 to sample #11, a proportional increase in the number of nucleocapsid barcodes was detected by the machine. As shown in FIG. 10, the R² value of the linear function is 0.995. R² is a measure of how much of the variation in the dependent variable (i.e., the number of barcodes detected) can be explained by variation in the independent variable (i.e., the number of antibodies in the sample). An R² value that is close to 1 indicates an extremely linear relationship between the two variables.

TABLE 2
Detection and quantification of Nucleocapsid barcodes

| | | Nucleocapsid Barcodes Detected | | |
|---|---|---|---|---|
| Sample | | Run | Run | Run |
| # | Sample Name | 11/11 | 11/12 | 11/19 |
| 1 | Spike 0 Nuc 10,000 | 34,312 | 32,460 | 35,074 |
| 2 | Spike 100 Nuc 10,000 | 36,755 | 34,641 | 41,761 |
| 3 | Spike 1,000 Nuc 10,000 | 40,139 | 35,818 | 36,382 |
| 4 | Spike 10,000 Nuc 10,000 | 33,836 | 32,643 | 33,106 |
| 5 | Spike 100,000 Nuc 10,000 | 32,439 | 38,495 | 40,453 |
| 6 | Spike 1,000,000 | 40,460 | 41,211 | 39,334 |
| | Nuc 10,000 | | | |
| 7 | Spike 10,000 Nuc 0 | 187 | 94 | 10 |
| 8 | Spike 10,000 Nuc 100 | 378 | 400 | 348 |
| 9 | Spike 10,000 Nuc 1,000 | 3,520 | 3,799 | 3,812 |
| 10 | Spike 10,000 Nuc 10,000 | 39,368 | 38,075 | 40,678 |
| 11 | Spike 10,000 Nuc 100,000 | 325,514 | 364,448 | 379,048 |
| 12 | Spike 10,000 Nuc | 492,256 | 492,256 | 492,256 |
| | 1,000,000 | | | |

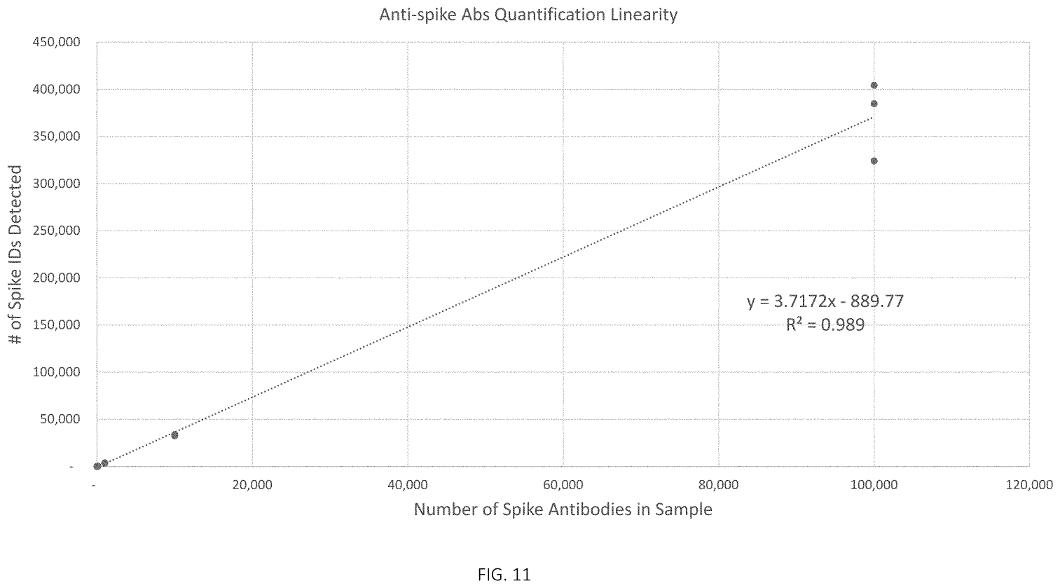

Table 3 shows the detection and quantification of Spike barcodes from S-BLAs bound by anti-spike antibodies. Results also show an extremely linear relationship between the number of anti-spike antibodies in the sample and the number of spike barcodes measured by the sequencer, i.e., as the number of anti-spike antibodies increased from sample #1 to sample #5, a proportional increase in the number of spike barcodes was detected by the machine. As shown in FIG. 11, the R² value of the linear function is 0.989.

TABLE 3
Detection and quantification of Spike barcodes

| | | Spike Barcodes Detected | | |
|---|---|---|---|---|
| Sample | | Run | Run | Run |
| # | Sample Name | 11/11 | 11/12 | 11/19 |
| 1 | Spike 0 Nuc 10,000 | 44 | 150 | 10 |
| 2 | Spike 100 Nuc 10,000 | 381 | 360 | 431 |
| 3 | Spike 1,000 Nuc 10,000 | 4,019 | 3,587 | 3,644 |
| 4 | Spike 10,000 Nuc 10,000 | 33,836 | 32,643 | 33,106 |
| 5 | Spike 100,000 Nuc 10,000 | 324,230 | 384,792 | 404,366 |
| 6 | Spike 1,000,000 | 418,918 | 484,566 | 492,073 |
| | Nuc 10,000 | | | |
| 7 | Spike 10,000 Nuc 0 | 41,908 | 32,576 | 34,548 |
| 8 | Spike 10,000 Nuc 100 | 37,491 | 39,682 | 34,475 |
| 9 | Spike 10,000 Nuc 1,000 | 35,092 | 37,886 | 38,017 |
| 10 | Spike 10,000 Nuc 10,000 | 39,368 | 38,075 | 40,678 |
| 11 | Spike 10,000 Nuc 100,000 | 32,562 | 36,455 | 37,915 |
| 12 | Spike 10,000 Nuc | 34,745 | 41,244 | 34,745 |
| | 1,000,000 | | | |

A saturation effect was observed when antibody levels reached above 100,000, causing a decrease in linearity (samples #6 and #12 in Table 2 and Table 3). However, because 1,000,000 antibodies in a microliter is a far higher concentration than what exists in nature, this limit in antibody range was deemed irrelevant for clinical application purposes.

### Example 4—Diagnostic Testing and Medical Guidance

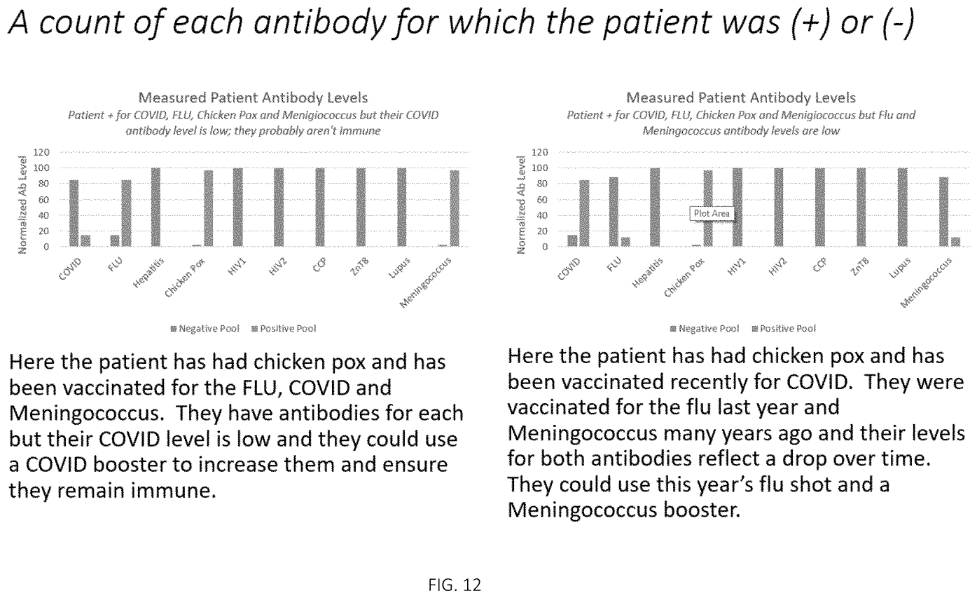

FIG. 12 provides a prophetic example of how methods and compositions of the disclosure could be used to guide and inform medical care. The test described in FIG. 12 measures antibody levels for infectious diseases including SARS CoV-2, HIV and Hepatitis. Two exemplary histograms are shown. In the histogram on the left, patient #1 has antibodies for the Flu, Chicken Pox and Meningococcus but their level of SARS-CoV-2 antibodies is low compared to the others, suggesting patient #1 could benefit from a SARS-CoV-2 vaccine boost. The second patient shown in the histogram on the right has high levels of SARS-CoV-2 antibodies, but their Flu antibodies are low, suggesting patient #2 could benefit from a Flu vaccine boost. Testing either patient with a qualitative ELISA test for FLU and SARS-CoV-2 antibodies would not provide useful information, as both patients would likely test positive for both antibodies, falsely indicating that they are immune to both diseases.

### Example 5—Proof of Concept in an 8-BLA System

### I. Oligo Design

Oligos were designed for 8 different antigens, each including, from 5′ to 3′, a spacer, a restriction enzyme cleavage site (HAEIII), a forward primer (FP) binding site, an Antibody Identification sequence (AIS) unique for each BLA, and a reverse primer (RP) binding site. The design of the 8 oligos is shown in Table 4.

TABLE 4
Design of 8 oligos

| | SEQ ID NOs | | | | | |
|---|---|---|---|---|---|---|
| | | | FP | | RP | Full- |
| | | Cleavage | Binding | | Binding | length |
| 5′ Antigen | Spacer | Site | Site | AIS | Site | Oligo |
| SARS-CoV2 Spike | 15 | 3 | 16 | 6 | 19 | 1 |
| SARS-CoV2 Nucleocapsid | 15 | 3 | 17 | 7 | 18 | 2 |
| HCMV Glycoprotein B | 15 | 3 | 16 | 20 | 19 | 26 |
| EBV Glycoprotein gp350 | 15 | 3 | 17 | 21 | 18 | 27 |
| Influenza A Hemagglutinin | 15 | 3 | 16 | 22 | 19 | 28 |
| Influenza B Hemagglutinin | 15 | 3 | 17 | 23 | 18 | 29 |
| Varicella-Zoster | 15 | 3 | 16 | 24 | 19 | 30 |
| Heterodimer gE/gI | | | | | | |
| hMPV B Glycoprotein G | 15 | 3 | 17 | 25 | 18 | 31 |

The AIS is unique for each oligo. Oligos were ordered from IDT in 2 μmole quantities with HPLC purification.

### II. Synthesis of Barcode Labelled Antigens (BLAs)

The following 8 antigens were obtained:

1. SARS-CoV-2 (2019-nCoV) Spike S1-His Recombinant Protein (HPLC-verified) (Sino Biological Cat: 40591-VO8H). This antigen allows detection of SARS-CoV-2 Spike antibodies generated by the immune system in response to infection or vaccine.

2. SARS-CoV-2 (2019-nCoV) Nucleocapsid-His Recombinant Protein (Sino Biological Cat: 40588-V07E). This antigen allows detection of SARS-CoV-2 Nucleocapsid antibodies generated in response to infection. These Nucleocapsid would not be generated in response to a SARS-CoV-2 vaccine and thus can be used to determine if a person's immunity is obtained from an infection or from vaccination.

3. Influenza A [A/Hawaii/70/2019 (H1N1)pdm09-like virus] Hemagglutinin (HA), His-Tag (Native Antigen Company #REC31884-100). This antigen allows detection of antibodies generated by the 2020-2021 flu vaccine.

4. Influenza B [B/Washington/02/2019 (B/Victoria lineage)-like virus] Hemagglutinin (HA), His-Tag (Native Antigen Company #REC31953-100). This antigen allows detection of antibodies generated by the 2020-2021 flu vaccine.

5. Varicella-Zoster Virus Heterodimer gE/gI (HEK293), His-tag (Native Antigen Company #REC31907-100). This antigen allows detection of the Varicella-Zoster Virus and serves as an internal control.

6. Human Metapneumovirus (hMPV) B Glycoprotein G, His-Tag (HEK293) (Native Antigen Company #REC31948-100). This antigen allows detection of hMPV and serves as an internal control.

7. Human cytomegalovirus (HCMV) Glycoprotein B/gB Protein (His Tag) (Sino Biological 10202-V08H1). This antigen allows detection of HCMV and serves as an internal control.

8. Epstein-Barr virus (Herpesvirus 4) EBV Glycoprotein gp350/EBV GP350 Protein (His Tag) (Sino Biological 40373-VO8H). This antigen allows detection of EBV and serves as an internal control.

Because most people in the US population have antibodies to most of the viruses in No. 5-No. 8 listed above, these internal controls were designed to serve as “immune benchmarks” to allow normalization of SARS-CoV-2 antibody levels within the context of an individual's immune system.

Each oligo of the 8 oligos described above was conjugated to its corresponding antigen using the SoluLINK Protein—Oligonucleotide Conjugation Kit (Vector® Laboratories Cat. No. S-9011) according to the manufacturer's instructions. Protein-oligo conjugation kits from Abcam (cat. No. ab218260) and Novus Biologicals (Cat. No. 425-0300) have also been used to synthesize BLAs with equal effectiveness.

After conjugation, all 8 BLAs were pooled in equal molar concentration (2 nm/mL) in 1× Phosphate-Buffered Saline (PBS)(137 mM NaCl; 2.7 mM KCl; 10 mM Na2HPO4; 1.8 mM KH2PO4; pH 7.4) to make the BLA Mix solution.

### III. Generation of Reference Samples

Synthetic “reference samples” were made to mimic a biological sample comprising antibodies against SARS-CoV-2 obtained from a human. The reference samples were generated according to the following table by diluting SARS-CoV-2 (2019-nCoV) Spike 51 Antibody, Rabbit MAb (Sino Biological Cat: 40150-R007) and SARS-CoV-2 (2019-nCoV) Nucleocapsid Antibody, Rabbit MAb (Sino Biological Cat: 40143-R019) in 2% (w/v) Bovine Serum Albumin (BSA).

TABLE 5
Reference Samples

| | | Conc. Spike Ab | Conc. Nucleocapsid Ab |
|---|---|---|---|
| # | Sample Name | (pg/mL) | (pg/mL) |
| 1 | 0X Spk, 1X Nuc | 0 | 200 |
| 2 | 0.25X Spk, 1X Nuc | 50 | 200 |
| 3 | 1X Spk, 1X Nuc | 200 | 200 |
| 4 | 4X Spk, 1X Nuc | 800 | 200 |

### IV. Generation of Captured Barcodes

The barcodes corresponding to each sample were captured according to the following procedure.

1. Binding Abs to BLAs to generate BLA-Ab complexes. In a 96-well, 200 μL/well PCR plate, 25 μL of each reference sample was added to a separate well, followed by the addition of 25 μL of BLA Mix Solution to each well. The resulting mixture was incubated at room temperature (RT) for 1 hour.

2. Binding biotinylated antiAbs to BLA-Ab complexes to generate BLA-Ab-antiAb complexes: 50 μL of Goat Anti-Rabbit IgG Secondary Antibody (Biotin) (Sino Biological Cat: SSA011) at 2 nm/mL in BSA was added to each well and incubated at RT for 1 hour.

3. Magnetic selection for BLA-Ab-antiAb complexes.
a. 100 μL of MagnaLINK Streptavidin Beads (Vector Labs M-1003-010) at 10 mg/mL in Wash Buffer (50 mM Tris-HCl, 150 mM NaCl, 0.05% Tween 20, pH 8.0) were added to the well next to each sample well. The plate was placed on a magnet and rotated for 2 minutes to pellet the beads.
b. The supernatant was removed and discarded without disturbing the bead pellets. The plate was removed from the magnet and 200 μL of Wash Buffer was added to each sample. The pellets were resuspended in Wash Buffer and the plate was returned to the magnet for 2 minutes.
c. Step ii was repeated once.
d. 100 μL of 2× Wash Buffer (100 mM Tris-HCl, 300 mM NaCl, 0.1% Tween 20, pH 8.0) and 100 μL of the sample from step 2 was added to each corresponding well. The solutions were mixed well and incubated at RT for 1 hour.
e. The plate was returned to magnet for 2 minutes. The supernatant was removed and discarded without disturbing the bead pellets. The plate was removed from magnet and 200 μL of Wash Buffer was added to each well to resuspend the pellets.

4. Cleaving the DNA barcodes.
a. To the next well beside each sample well, 19 μL of the corresponding sample from step 3(e) was added, followed by 2 μL of 10× FastDigest® buffer and 1 μL of FastDigest BsuRI enzyme (ThermoFishher Catalog number: FD0154).
b. The wells were mixed gently then incubated at 37° C. for 5 minutes.

### V. Sequencing of Captured Barcodes

Illumina® sequencing adapters with sample index sequences were used in PCR to allow multiplexing of the captured barcodes.

The following procedure was used to generate a DNA library from the captured DNA barcodes.

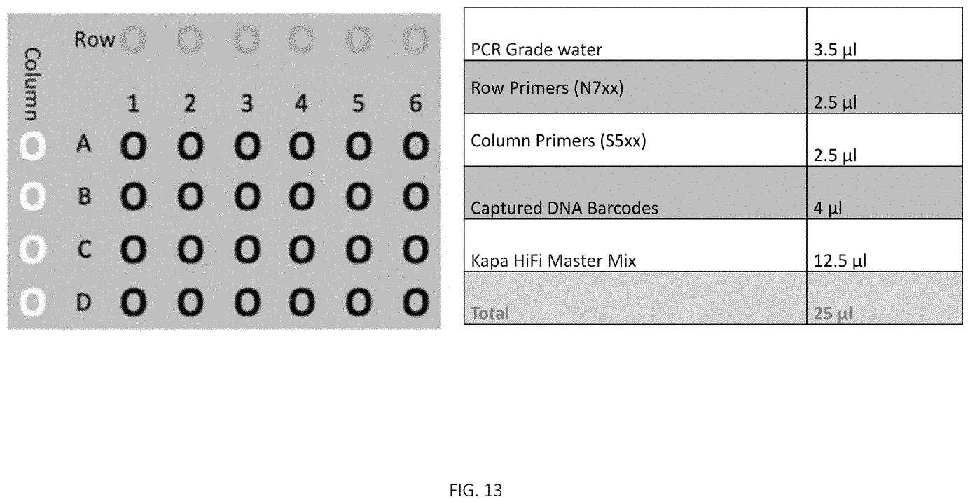

1. An Index Plate was set up according to FIG. 13. The Nextera Index Kit (Illumina®) containing Row Primers (Index 1 Read of Nextera Index Kit) and Column Primers (Index 2 Read of Nextera Index Kit) were used. Kapa HiFi Master Mix (Roche Material Number: 7958927001) was used.

2. The solution in each well of the Index Plate was gently mixed, and the resulting mixtures were centrifuged for about 5 seconds.

3. The mixtures of step 2 were amplified on a thermal cycler using the following PCR program:
a. 95° C. for 3 minutes
b. 10 cycles of:
   i. 98° C. for 30 seconds
   ii. 55° C. for 30 seconds
   iii. 72° C. for 30 seconds
c. 72° C. for 5 minutes
d. Hold at 4° C.

Following PCR, DNA purification was performed using the following procedure.

1. 20 μL of AMPure XP beads were added to each well in a purification plate.

2. 30 μL of each sample was added to each well of the purification plate, gently mixed, and incubate at room temperature without shaking for 5 minutes.

3. The purification plate was placed on a magnetic stand for 5 minutes or until the supernatant has cleared.

4. 40 μL of the supernatant in each well was removed and discarded.

5. With the purification plate on the magnetic stand, the beads were washed with freshly prepared 80% ethanol as follows:
a. 200 μL of freshly prepared 80% ethanol was added to each sample well.
b. The plate was incubated on the magnetic stand for 30 seconds.
c. The supernatant was removed and discarded.

6. With the purification plate on the magnetic stand, a second ethanol wash was performed as follows:
a. 200 μL of freshly prepared 80% ethanol was added to each sample well.
b. The plate was incubated on the magnetic stand for 30 seconds.
c. The supernatant was removed and discarded.
d. Excess ethanol was removed.

7. With the purification plate still on the magnetic stand, the beads were air-dried for 5 minutes.

8. The purification plate was removed from the magnetic stand and 40 μL of 10 mM Tris pH 8.0 was added to each well of the purification plate. The wells were gently mixed to fully resuspend the beads, followed by incubation at room temperature for 2 minutes.

9. The purification plate was placed on the magnetic stand for 2 minutes or until the supernatant has cleared.

10. 32 μL of the supernatant from each well of the purification plate was transferred to pre-labeled empty strip tubes and stored for future use.

Following DNA purification, each of the generated libraries were quantified using the following procedure.

1. Buffered Pico Green solution was prepared by adding 5373 μL of Qubit Buffer solution to 27 μL of Pico Green reagent.

2. 198 μL of prepared buffered Pico Green solution was added to each of the DNA sample assay tubes; 190 μL of prepared buffered Pico Green solution was added to each of the standard assay tubes.

3. 2 μL of each DNA sample to its corresponding assay tube; 10 μL of each standard sample was added to its corresponding assay tube. Qubit dsDNA HS Standard (Fisher Scientific) were used as standard samples.

4. All tubes were vortexed for 2-3 seconds and incubated for 2 minutes at room temperature.

5. Measurements were made using a Qubit Fluorometer.

The library concentration determined using the method above will be used to calculate the amount of TE solution to be added for normalization. After normalization, the final concentration of each sample was 4 nM.

The normalized samples were denatured and prepared for sequencing using the following procedure.

1. 5 μL of each normalized 4 nM DNA sample was combined with 5 μL of 0.2 N NaOH in a microcentrifuge tube.

2. Each sample tube was vortexed for 5-10 seconds, centrifuged for 30 seconds, and incubate at room temperature for 5 minutes to denature the DNA strands.

3. 990 μL of pre-chilled HT1 Hybridization Buffer (Illumina) was added to the tube containing the denatured DNA and vortex briefly. This resulted in a 20 pM denatured library in 1 mM NaOH.

4. The 20 pM DNA was further diluted by combining 570 μL of 20 pM DNA with 30 μL of Phi X Control (Illumina) in a microcentrifuge tube.

5. The tube was vortexed briefly and placed on ice until ready to load on to a MiSeq reagent cartridge.

The above samples were sequenced using the Illumina MiSeq system following manufacturer's instructions.

### VI. Results

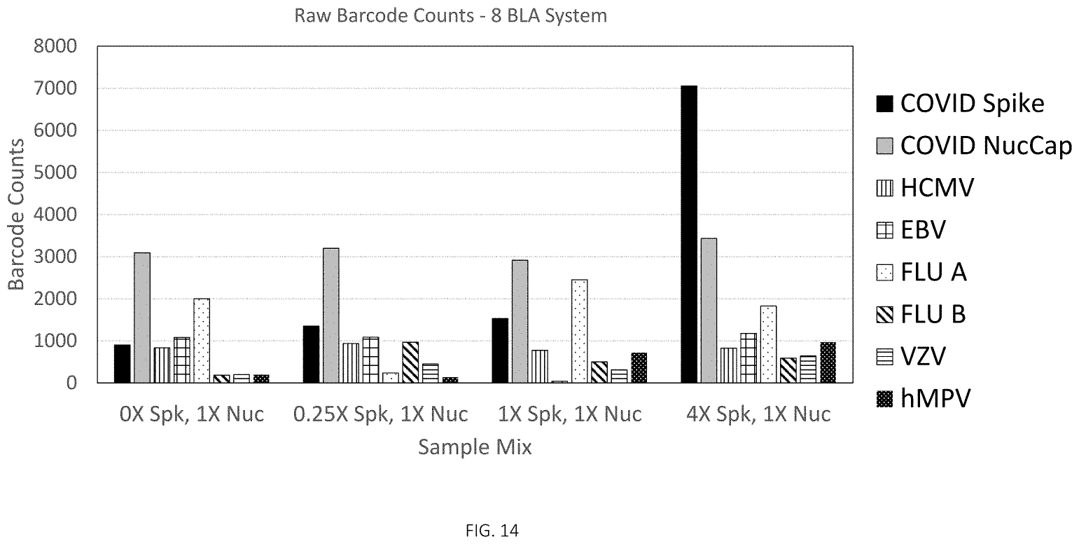

The sequence reads generated were filtered to include only exact matches to the AISs imbedded in the BLA molecules. The number of each AIS was calculated; the sequencing results are summarized in FIG. 14 and Table 6.

TABLE 6
Results of Barcode Sequencing

| | COVID | COVID | | | | | | |
|---|---|---|---|---|---|---|---|---|
| SAMPLE | Spike | NucCap | HCMV | EBV | FLU A | FLU B | VZV | hMPV |
| 0X Spk, 1X Nuc | 900 | 3093 | 836 | 1082 | 2001 | 185 | 200 | 201 |
| 0.25X Spk, 1X Nuc | 1350 | 3198 | 937 | 1088 | 241 | 967 | 451 | 141 |
| 1X Spk, 1X Nuc | 1532 | 2915 | 775 | 42 | 2451 | 501 | 312 | 719 |
| 4X Spk, 1X Nuc | 7055 | 3437 | 827 | 1178 | 1829 | 591 | 641 | 969 |

The raw barcode counts showed a positive correlation between the number of Spike barcodes read by the sequencer and the concentration of Spike antibodies in the sample: increasing from 900 to 1350, 1532 and 7055 as the concentration of Spike antibody increased from 0 to 0.025×, 1×, and 4×, respectively. In each of the four reference samples, the concentration of Nucleocapsid antibodies is the same (1× concentration), and a consistent number of NucCap barcodes was observed across the four samples: 3093, 3198, 2915, 3437, respectively.

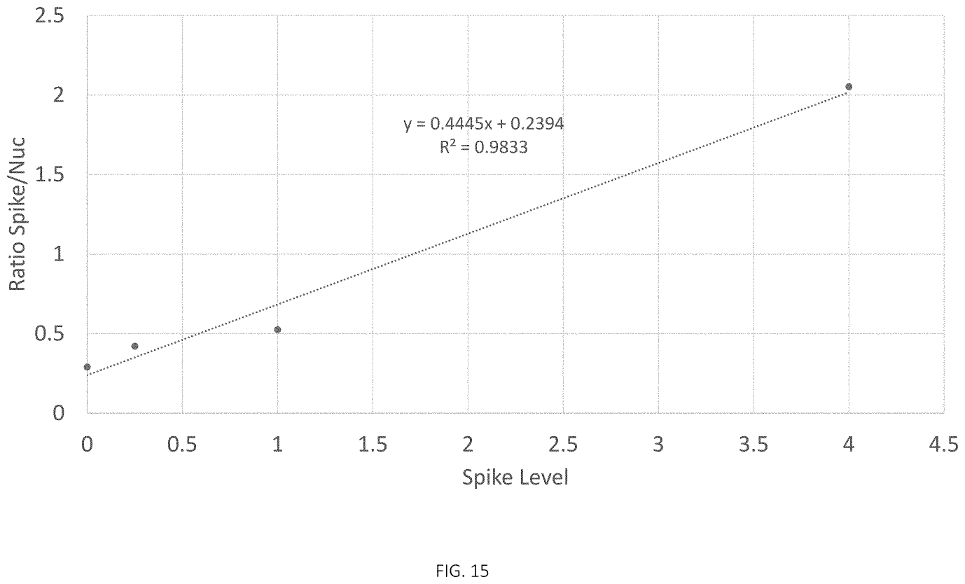

Due to background noise originating from the non-COVID BLAs (HCMV, EBV, FLU A, FLU B, VZV, and hMPV), the spike barcode counts were normalized to the nucleocapsid counts to provide a more linear relation to the Spike Ab concentration as reflected in Table 7 and FIG. 15.

TABLE 7
Normalization of Spike barcodes

| | Spike Conc. | Spike #/Nuc # |
|---|---|---|
| 0X Spk, 1X Nuc | 0 | 0.290983 |
| 0.25X Spk, 1X Nuc | 0.25 | 0.422132 |
| 1X Spk, 1X Nuc | 1 | 0.525642 |
| 4X Spk, 1X Nuc | 4 | 2.052623 |

Normalizing the number of Spike barcode reads to the number of Nucleocapsid barcode reads provided a tight correlation to the Spike concentration in the samples and showed the benefit of using the level of one antibody as an internal control to quantify another antibody in a given sample.

SEQ ID NO: 1
tcatGGCCTCGTCGGCAGCGTCAGATGTGTATAAGAGACAGagacgatta
agaccttggttaattgCTGTCTCTTATACACATCTCCGAGCCCACGAGAC
SEQ ID NO: 2
tcatGGCCCTGTCTCTTATACACATCTGACGCTGCCGACGAtcctgaagt
atcaaaatcaatctgaGTCTCGTGGGCTCGGAGATGTGTATAAGAGACAG
SEQ ID NO: 3
GGCC
SEQ ID NO: 4
TCGTCGGCAGCGTC
SEQ ID NO: 5
GTCTCGTGGGCTCGG
SEQ ID NO: 6
AGACGATTAAGACCTTGGTTAATTG
SEQ ID NO: 7
TCCTGAAGTATCAAAATCAATCTGA
SEQ ID NO: 8
AATGATACGGCGACCACCGAGATCTACACCTCTCTATTCGTCGGCAGCGT
C
SEQ ID NO: 9
CAAGCAGAAGACGGCATACGAGATTCGCCTTAGTCTCGTGGGCTCGG
SEQ ID NO: 10
AATGATACGGCGACCACCGA
SEQ ID NO: 11
CAAGCAGAAGACGGCATACGAGAT
SEQ ID NO: 12
AGATGTGTATAAGAGACAG
SEQ ID NO: 13
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT
SEQ ID NO: 14
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFYAEGSRGGSQASSRSSSRSRNSSRNSTPG
SSRGTSPARMAGNGGDAALALLLLDRLNQLESKMSGKGQQQQGQTVTKKS
AAEASKKPRQKRTATKAYNVTQAFGRRGPEQTQGNFGDQELIRQGTDYKH
WPQIAQFAPSASAFFGMSRIGMEVTPSGTWLTYTGAIKLDDKDPNFKDQV
ILLNKHIDAYKTFPPTEPKKDKKKKADETQALPQRQKKQQTVTLLPAADL
DDFSKQLQQSMSSADSTQA
SEQ ID NO: 15
tcat
SEQ ID NO: 16
TCGTCGGCAGCGTCAGATGTGTATAAGAGACAG
SEQ ID NO: 17
CTGTCTCTTATACACATCTGACGCTGCCGACGA
SEQ ID NO: 18
GTCTCGTGGGCTCGGAGATGTGTATAAGAGACAG
SEQ ID NO: 19
CTGTCTCTTATACACATCTCCGAGCCCACGAGAC
SEQ ID NO: 20
cattatcagcatccgctttgagtgc
SEQ ID NO: 21
cagctcaattcggtttgcgtccggc
SEQ ID NO: 22
aataaatgagtgaagggctcggtag
SEQ ID NO: 23
ctcaagagctatagtaactgtagac
SEQ ID NO: 24
caatctgattgcttcactgcaccgg
SEQ ID NO: 25
catcagactacggcactaagcggca
SEQ ID NO: 26
tcatGGCCTCGTCGGCAGCGTCAGATGTGTATAAGAGACAGcattatcag
catccgctttgagtgcCTGTCTCTTATACACATCTCCGAGCCCACGAGAC
SEQ ID NO: 27
tcatGGCCCTGTCTCTTATACACATCTGACGCTGCCGACGAcagctcaat
tcggtttgcgtccggcGTCTCGTGGGCTCGGAGATGTGTATAAGAGACAG
SEQ ID NO: 28
tcatGGCCTCGTCGGCAGCGTCAGATGTGTATAAGAGACAGcattatcag
catccgctttgagtgcCTGTCTCTTATACACATCTCCGAGCCCACGAGAC
SEQ ID NO: 29
tcatGGCCCTGTCTCTTATACACATCTGACGCTGCCGACGActcaagagc
tatagtaactgtagacGTCTCGTGGGCTCGGAGATGTGTATAAGAGACAG
SEQ ID NO: 30
tcatGGCCTCGTCGGCAGCGTCAGATGTGTATAAGAGACAGcaatctgat
tgcttcactgcaccggCTGTCTCTTATACACATCTCCGAGCCCACGAGAC
SEQ ID NO: 31
tcatGGCCCTGTCTCTTATACACATCTGACGCTGCCGACGAcatcagact
acggcactaagcggcaGTCTCGTGGGCTCGGAGATGTGTATAAGAGACAG

1. A method of identifying and/or quantifying one or more binding members in a test sample capable of binding to each of one or more targets, comprising:
a. contacting a test sample comprising binding members with a composition comprising at least one barcode-linked antigen (BLA), wherein each BLA comprises a barcode moiety linked to an antigen of a target, wherein binding members present in the test sample bind to antigens of the BLAs, thereby forming a mixture comprising BLA/binding member complexes;
b. optionally, isolating the BLA/binding member complexes from the mixture; and
c. determining the identity and quantity of the barcode moiety of each isolated BLA/binding member complex;
wherein the identity of each barcode moiety indicates the identity of its linked antigen and/or target, and the quantity of each barcode moiety indicates the quantity of binding members present in the test sample that bind its linked antigen and/or target,
thereby identifying and/or quantifying the binding members in the test sample capable of binding to each of one or more targets.

2. The method of claim 1, wherein the test sample is a biological sample obtained from a subject.

3-4. (canceled)

5. The method of claim 1, wherein the binding members comprise antibodies.

6. The method of claim 1, wherein at least one target is a pathogen.

7-8. (canceled)

9. The method of claim 1, wherein the composition comprises a pool of BLAs.

10. The method of claim 9, wherein the pool of BLAs comprises BLAs comprising different antigens.

11-14. (canceled)

15. The method of claim 1, wherein at least one antigen comprises a polypeptide, or an immunogenic fragment, variant, or epitope thereof.

16. (canceled)

17. The method of claim 1, wherein the method further comprises separating the barcode moiety of each BLA/binding member complex from its linked antigen.

18. The method of claim 1, wherein each antigen is attached to its linked barcode moiety by a bond or linker, wherein the bond or linker is cleavable or breakable.

19-23. (canceled)

24. The method of claim 1, wherein each barcode moiety comprises an oligonucleotide, a peptide, a nano-structure, a fluorescent bead, an isobaric mass tag, or a combination thereof.

25-28. (canceled)

29. The method of claim 128, wherein each barcode moiety comprises an oligonucleotide, wherein each oligonucleotide comprises
i. a unique sequence;
ii. one or more primer binding sites; and, optionally,
iii. a cleavage site.

30. The method of claim 29, wherein the antigen of each BLA is identifiable by the unique sequence.

31. The method of claim 29, wherein the target from which the antigen of each BLA was derived is identifiable by the unique sequence.

32. The method of claim 29, wherein each oligonucleotide is about the same length.

33-36. (canceled)

37. The method of claim 29, wherein a unique sequence is assigned to each different antigen.

38. (canceled)

39. The method of any one claim 29, wherein determining the identity and quantity of the oligonucleotide of each isolated BLA/binding member complex comprises:
amplifying the oligonucleotides with primers to generate one or more sequencing-ready libraries;
sequencing the one or more libraries to identify the sequence of each oligonucleotide; and
quantifying each oligonucleotide.

40-42. (canceled)

43. The method of claim 39, wherein each primer used for amplification comprises a sequence complementary to the primer binding site of each oligonucleotide and an adaptor sequence compatible with the sequencing method.

44-45. (canceled)

46. A kit used to quantify binding members in a test sample, optionally according to the method of claim 1, the kit comprising a first vessel comprising a composition comprising at least one barcode-linked antigen (BLA), wherein each BLA comprises a barcode moiety attached to an antigen of a target.

47-86. (canceled)

87. A composition comprising at least one barcode-linked antigen (BLA), wherein each BLA comprises a barcode moiety linked to an antigen of a target;
wherein the barcode moiety can be quantified; and
wherein the barcode moiety can identify its linked antigen.

88-125. (canceled)

126. A method of producing the composition of claim 87, comprising attaching the barcode moiety to the antigen.


SEQUENCE LISTING (31 sequences)

SEQ ID NO: 1 - DNA, 100 bp, Artificial Sequence
  Full-length oligonucleotide (spike BLA)
tcatggcctcgtcggcagcgtcagatgtgtataagagacagagacgattaagaccttggttaattgctgtctcttatacacatctccgagcccacgagac

SEQ ID NO: 2 - DNA, 100 bp, Artificial Sequence
  Full-length oligonucleotide (Nuc BLA)
tcatggccctgtctcttatacacatctgacgctgccgacgatcctgaagtatcaaaatcaatctgagtctcgtgggctcggagatgtgtataagagacag

SEQ ID NO: 3 - DNA, 4 bp, Artificial Sequence
  Restriction enzyme cleavage site HaeIII
ggcc

SEQ ID NO: 4 - DNA, 14 bp, Artificial Sequence
  Primer binding site 1 for index amplification
tcgtcggcagcgtc

SEQ ID NO: 5 - DNA, 15 bp, Artificial Sequence
  Primer binding site 2 for index amplification
gtctcgtgggctcgg

SEQ ID NO: 6 - DNA, 25 bp, Artificial Sequence
  Spike AIS (unique sequence)
agacgattaagaccttggttaattg

SEQ ID NO: 7 - DNA, 25 bp, Artificial Sequence
  Nuc AIS (unique sequence)
tcctgaagtatcaaaatcaatctga

SEQ ID NO: 8 - DNA, 51 bp, Artificial Sequence
  Amplification primer 1
aatgatacggcgaccaccgagatctacacctctctattcgtcggcagcgtc

SEQ ID NO: 9 - DNA, 47 bp, Artificial Sequence
  Amplification primer 2
caagcagaagacggcatacgagattcgccttagtctcgtgggctcgg

SEQ ID NO: 10 - DNA, 20 bp, Artificial Sequence
  P5
aatgatacggcgaccaccga

SEQ ID NO: 11 - DNA, 24 bp, Artificial Sequence
  p7
caagcagaagacggcatacgagat

SEQ ID NO: 12 - DNA, 19 bp, Artificial Sequence
  Sequencing primer binding site
agatgtgtataagagacag

SEQ ID NO: 13 - protein, 1273 aa, Artificial Sequence
  spike antigen
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - protein, 419 aa, Artificial Sequence
  Nuc antigen
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 15 - DNA, 4 bp, Artificial Sequence
  Spacer
tcat

SEQ ID NO: 16 - DNA, 33 bp, Artificial Sequence
  Forward Primer
tcgtcggcagcgtcagatgtgtataagagacag

SEQ ID NO: 17 - DNA, 33 bp, Artificial Sequence
  Forward Primer Complement
ctgtctcttatacacatctgacgctgccgacga

SEQ ID NO: 18 - DNA, 34 bp, Artificial Sequence
  Reverse Primer
gtctcgtgggctcggagatgtgtataagagacag

SEQ ID NO: 19 - DNA, 34 bp, Artificial Sequence
  Reverse Primer Complement
ctgtctcttatacacatctccgagcccacgagac

SEQ ID NO: 20 - DNA, 25 bp, Artificial Sequence
  AIS for HCMV Glycoprotein B
cattatcagcatccgctttgagtgc

SEQ ID NO: 21 - DNA, 25 bp, Artificial Sequence
  AIS for EBV Glycoprotein gp350
cagctcaattcggtttgcgtccggc

SEQ ID NO: 22 - DNA, 25 bp, Artificial Sequence
  AIS for Influenza A Hemagglutinin
aataaatgagtgaagggctcggtag

SEQ ID NO: 23 - DNA, 25 bp, Artificial Sequence
  AIS for Influenza B Hemagglutinin
ctcaagagctatagtaactgtagac

SEQ ID NO: 24 - DNA, 25 bp, Artificial Sequence
  AIS for Varicella-Zoster Virus Heterodimer gE/gI
caatctgattgcttcactgcaccgg

SEQ ID NO: 25 - DNA, 25 bp, Artificial Sequence
  AIS for hMPV B Glycoprotein G
catcagactacggcactaagcggca

SEQ ID NO: 26 - DNA, 100 bp, Artificial Sequence
  full-length oligo (HCMV Glycoprotein B BLA)
tcatggcctcgtcggcagcgtcagatgtgtataagagacagcattatcagcatccgctttgagtgcctgtctcttatacacatctccgagcccacgagac

SEQ ID NO: 27 - DNA, 100 bp, Artificial Sequence
  full-length oligo (EBV Glycoprotein gp350 BLA)
tcatggccctgtctcttatacacatctgacgctgccgacgacagctcaattcggtttgcgtccggcgtctcgtgggctcggagatgtgtataagagacag

SEQ ID NO: 28 - DNA, 100 bp, Artificial Sequence; identical to SEQ ID NO: 26
  full-length oligo (Influenza A Hemagglutinin BLA)

SEQ ID NO: 29 - DNA, 100 bp, Artificial Sequence
  full-length oligo (Influenza B Hemagglutinin BLA)
tcatggccctgtctcttatacacatctgacgctgccgacgactcaagagctatagtaactgtagacgtctcgtgggctcggagatgtgtataagagacag

SEQ ID NO: 30 - DNA, 100 bp, Artificial Sequence
  full-length oligo (Varicella-Zoster Heterodimer gE/gI BLA)
tcatggcctcgtcggcagcgtcagatgtgtataagagacagcaatctgattgcttcactgcaccggctgtctcttatacacatctccgagcccacgagac

SEQ ID NO: 31 - DNA, 100 bp, Artificial Sequence
  full-length oligo (hMPV B Glycoprotein G BLA)
tcatggccctgtctcttatacacatctgacgctgccgacgacatcagactacggcactaagcggcagtctcgtgggctcggagatgtgtataagagacag